(19) United States
(12) Patent Application Publication
(10) Pub. No.: US 2011/0248251 A1
(43) Pub. Date: Oct. 13, 2011

(54) NITROGEN-CONTAINING HETEROCYCLIC DERIVATIVE AND ORGANIC ELECTROLUMINESCENCE ELEMENT USING NITROGEN-CONTAINING HETEROCYCLIC DERIVATIVE

(75) Inventors: Hiroshi Yamamoto, Chiba (JP); Jun Endo, Chiba (JP); Yuichiro Kawamura, Chiba (JP)

(73) Assignee: IDEMITSU KOSAN CO., LTD., TOKYO (JP)

(21) Appl. No.: 13/126,049

(22) PCT Filed: Oct. 28, 2009
(86) PCT No.: PCT/JP09/68472

§ 371 Date: Jul. 1, 2011

(30) Foreign Application Priority Data
Oct. 28, 2008 (JP) 2008-277105

(51) Int. Cl. H01L 51/54; C07D 471/14

(52) U.S. Cl. 257/40; 544/345; 257/E51.024

(57) ABSTRACT

Provided are a nitrogen-containing heterocyclic derivative having a specific structure containing a pyrazine skeleton in the center thereof, a hole injecting material or hole transporting material for an organic electroluminescence (EL) device, a light emitting material for an organic EL device, and an electron injecting material or electron transporting material for an organic EL device each containing the nitrogen-containing heterocyclic derivative, an organic EL device which includes one or a plurality of organic layers interposed between a cathode and an anode and in which at least one layer of the organic layers contains the nitrogen-containing heterocyclic derivative, and an apparatus including the organic EL device. The organic EL device shows high luminous brightness and high luminous efficiency even at a low voltage as compared with a conventional device.

### TECHNICAL FIELD

The present invention relates to a novel nitrogen-containing heterocyclic derivative, a material for an organic electroluminescence (EL) device using the derivative, and an organic EL device containing the material, in particular, an organic EL device that shows high luminous brightness and high luminous efficiency even at a low voltage.

### BACKGROUND ART

A large number of organic EL devices each using an organic substance have been developed because of their potential to find applications in solid emission-type, inexpensive, large-area, full-color display devices. In general, an EL device is formed of a light emitting layer and a pair of opposing electrodes between which the layer is interposed. Light emission is the following phenomenon. That is, upon application of an electric field to both electrodes, an electron is injected from a cathode side and a hole is injected from an anode side, and further, the electron recombines with the hole in the light emitting layer to produce an excited state, and energy generated upon return to a ground state from the excited state is radiated as light.

A conventional organic EL device is driven at a voltage higher than the voltage at which an inorganic light emitting diode is driven, and has lower luminous brightness and lower luminous efficiency than those of the inorganic light emitting diode. In addition, the properties of the device deteriorate so remarkably that the device cannot be put into practical use. Although a recent organic EL device has been gradually improved, high luminous brightness and high luminous efficiency at an even lower voltage have been requested of the device.

Aromatic amine derivatives have been conventionally known as hole injecting/transporting materials used in organic EL devices.

However, organic EL devices using those aromatic amine derivatives as their hole injecting/transporting materials are driven at high voltages, and hence the following material has been requested in recent years. A device using the material can be driven at a reduced voltage and can show improved efficiency.

To solve such problem, in, for example, Patent Literature 1, a specific substituent is incorporated into a skeleton having a specific hexaazatriphenylene structure to provide the skeleton with nature as a p-type semiconductor, and the resultant compound, which has electron accepting property, is used in a hole injecting region. As a result, the resultant device shows good performance, and a reduction in voltage at which the device is driven is achieved. However, the compound involves such problems that the compound crystallizes when energized for a long time period and has a short duration. In Patent Literature 2, a compound having the same specific hexaazatriphenylene structure as that of Patent Literature 1 is used, which is known to serve as an electron injecting material showing good electron injecting property. However, the compound also involves such problems that the compound crystallizes when energized for a long time period and has a remarkably short duration. As is known from Non Patent Literature 1 and Patent Literature 3, a compound having a dicyanopyrazine structure has electron accepting property, and hence can be used as a material for a field-effect transistor. However, the compound has involved such a problem that its application to an organic EL device or the like is significantly restricted because the compound absorbs a large quantity of light in a visible region.
[PTL 1] JP 3614405 B2
[PTL 2] JP 3571977 B2
[PTL 3] DE 10 2006 031 752
[NPL 1] Organic Letters, vol. 6, p. 2007, 2004.

### SUMMARY OF INVENTION

### Technical Problem

The present invention has been made to solve the above-mentioned problems, and an object of the present invention is to realize an organic EL device that shows high luminous brightness and high luminous efficiency even at a low voltage as compared with a conventional device.

### Solution to Problem

The inventors of the present invention have made extensive studies to achieve the object. As a result, the inventors have found that the object can be achieved by using a novel nitrogen-containing heterocyclic derivative having a specific structure containing a pyrazine skeleton represented by the following formula (1) in at least one layer of the organic compound layers of an organic EL device. Thus, the inventors have completed the present invention.

That is, the present invention provides the nitrogen-containing heterocyclic derivative represented by the following formula (1):

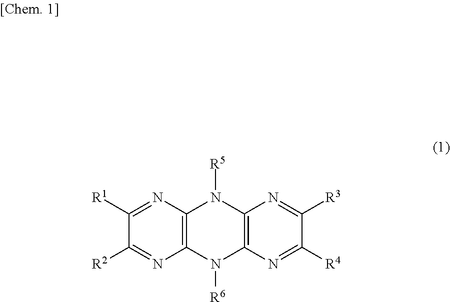

where:

R¹ to R⁴ each independently represent a hydrogen atom, a substituted or unsubstituted aryl group having 6 to 60 ring atoms, a substituted or unsubstituted heteroaryl group having 5 to 60 ring atoms, a substituted or unsubstituted alkyl group having 1 to 50 carbon atoms, a substituted or unsubstituted cycloalkyl group having 3 to 50 carbon atoms, a substituted or unsubstituted haloalkyl group having 1 to 50 carbon atoms, a substituted or unsubstituted aryloxycarbonyl group having 6 to 60 ring atoms, a substituted or unsubstituted alkoxycarbonyl group having 1 to 50 carbon atoms, a substituted or unsubstituted carbamoyl group, a substituted or unsubstituted arylcarbonyl group having 6 to 60 ring atoms, a substituted or unsubstituted alkylcarbonyl group having 1 to 50 carbon atoms, a substituted or unsubstituted arylsulfonyl group having 6 to 60 ring atoms, a substituted or unsubstituted alkylsulfonyl group having 1 to 50 carbon atoms, a substituted or unsubstituted arylsulfinyl group having 6 to 60 ring atoms, a substituted or unsubstituted alkylsulfinyl group having 1 to 50 carbon atoms, a halogen atom, a cyano group, or a nitro group, and adjacent groups of R¹ to R⁴ may be bonded to each other to form a ring structure; and

R⁵ and R⁶ each independently represent a substituted or unsubstituted aryl group having 6 to 60 ring atoms, a substituted or unsubstituted heteroaryl group having 5 to 60 ring atoms, a substituted or unsubstituted alkyl group having 1 to 50 carbon atoms, a substituted or unsubstituted cycloalkyl group having 3 to 50 carbon atoms, or a substituted or unsubstituted haloalkyl group having 1 to 50 carbon atoms.

The present invention also provides a hole injecting material or hole transporting material for an organic EL device, alight emitting material for an organic EL device, and an electron injecting material or electron transporting material for an organic EL device each containing the nitrogen-containing heterocyclic derivative.

The present invention also provides an organic EL device, including one or a plurality of organic layers interposed between a cathode and an anode, in which at least one layer of the organic layers contains the nitrogen-containing heterocyclic derivative of the present invention, and an apparatus, including the organic EL device.

### Advantageous Effects of Invention

The organic EL device using the nitrogen-containing heterocyclic derivative of the present invention shows high luminous brightness and high luminous efficiency even at a low voltage as compared with a conventional device.

### DESCRIPTION OF EMBODIMENTS

The present invention provides a nitrogen-containing heterocyclic derivative represented by the following formula (1).

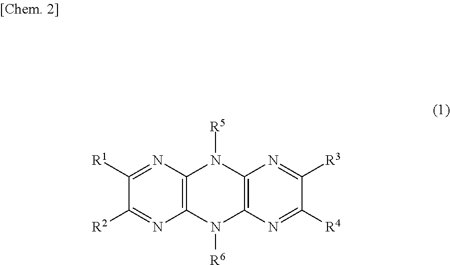

(In the formula: R¹ to R⁴ each independently represent a hydrogen atom, a substituted or unsubstituted aryl group having 6 to 60 ring atoms, a substituted or unsubstituted heteroaryl group having 5 to 60 ring atoms, a substituted or unsubstituted alkyl group having 1 to 50 carbon atoms, a substituted or unsubstituted cycloalkyl group having 3 to 50 carbon atoms, a substituted or unsubstituted haloalkyl group having 1 to 50 carbon atoms, a substituted or unsubstituted aryloxycarbonyl group having 6 to 60 ring atoms, a substituted or unsubstituted alkoxycarbonyl group having 1 to 50 carbon atoms, a substituted or unsubstituted carbamoyl group, a substituted or unsubstituted arylcarbonyl group having 6 to 60 ring atoms, a substituted or unsubstituted alkylcarbonyl group having 1 to 50 carbon atoms, a substituted or unsubstituted arylsulfonyl group having 6 to 60 ring atoms, a substituted or unsubstituted alkylsulfonyl group having 1 to 50 carbon atoms, a substituted or unsubstituted arylsulfinyl group having 6 to 60 ring atoms, a substituted or unsubstituted alkylsulfinyl group having 1 to 50 carbon atoms, a halogen atom, a cyano group, or a nitro group, and adjacent groups of R¹ to R⁴ may be bonded to each other to form a ring structure; and

R⁵ and R⁶ each independently represent a substituted or unsubstituted aryl group having 6 to 60 ring atoms, a substituted or unsubstituted heteroaryl group having 5 to 60 ring atoms, a substituted or unsubstituted alkyl group having 1 to 50 carbon atoms, a substituted or unsubstituted cycloalkyl group having 3 to 50 carbon atoms, or a substituted or unsubstituted haloalkyl group having 1 to 50 carbon atoms.)

The nitrogen-containing heterocyclic derivative represented by the formula (1) of the present invention is preferably one represented by the following formula (2), (3-a), (3-b), (3-c), (3-d), (3-e), (3-f), (3-g), (3-h), (4-a), or (4-b).

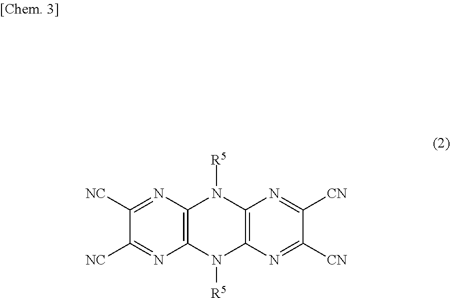

(R⁵ and R⁶ have the same meaning as that described above.)

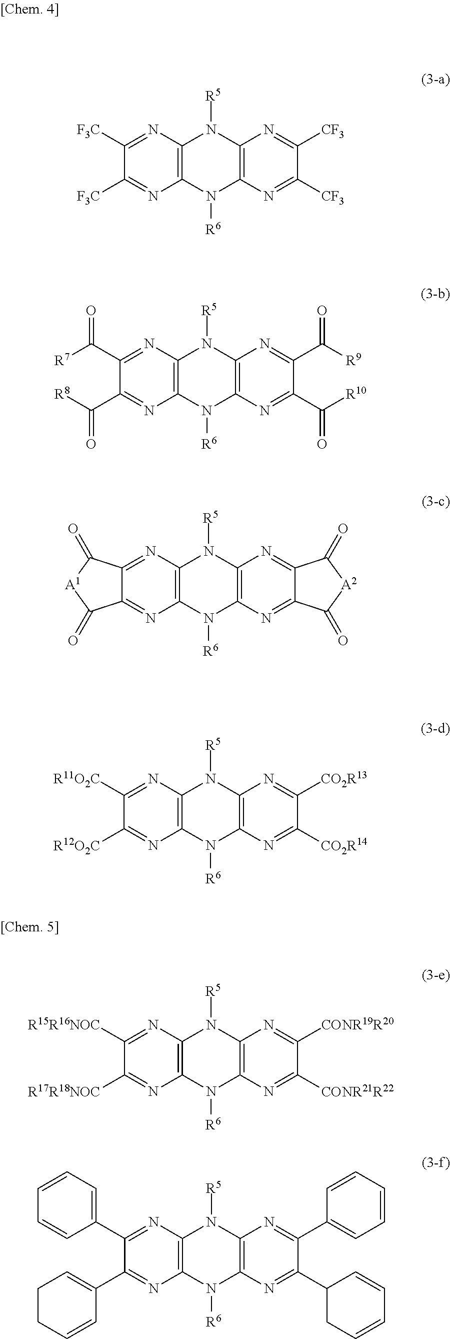

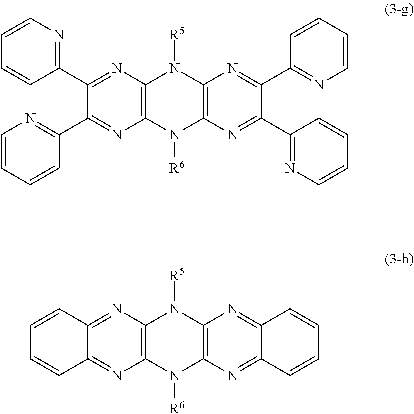

(In the formulae: R⁷ to R¹⁰ each independently represent a substituted or unsubstituted aryl group having 6 to 60 ring atoms, a substituted or unsubstituted heteroaryl group having 5 to 60 ring atoms, a substituted or unsubstituted alkyl group having 1 to 50 carbon atoms, a substituted or unsubstituted cycloalkyl group having 3 to 50 carbon atoms, or a substituted or unsubstituted haloalkyl group having 1 to 50 carbon atoms, and adjacent substituents may be bonded to each other to form a ring structure;

A¹ and A² each independently represent an oxygen atom or —NR′—, and R′ represents a substituted or unsubstituted arylene group having 6 to 60 ring atoms, a substituted or unsubstituted heteroarylene group having 5 to 60 ring atoms, a substituted or unsubstituted alkylene group having 1 to 50 carbon atoms, a substituted or unsubstituted cycloalkylene group having 3 to 50 carbon atoms, or a substituted or unsubstituted haloalkylene group having 3 to 50 carbon atoms;

R¹¹ to R¹⁴ each independently represent a substituted or unsubstituted aryl group having 6 to 60 ring atoms, a substituted or unsubstituted heteroaryl group having 5 to 60 ring atoms, a substituted or unsubstituted alkyl group having 1 to 50 carbon atoms, a substituted or unsubstituted cycloalkyl group having 3 to 50 carbon atoms, or a substituted or unsubstituted haloalkyl group having 1 to 50 carbon atoms; and

R¹⁵ to R²² each independently represent a hydrogen atom, a substituted or unsubstituted aryl group having 6 to 60 ring atoms, a substituted or unsubstituted heteroaryl group having 5 to 60 ring atoms, a substituted or unsubstituted alkyl group having 1 to 50 carbon atoms, a substituted or unsubstituted cycloalkyl group having 3 to 50 carbon atoms, or a substituted or unsubstituted haloalkyl group having 1 to 50 carbon atoms.)

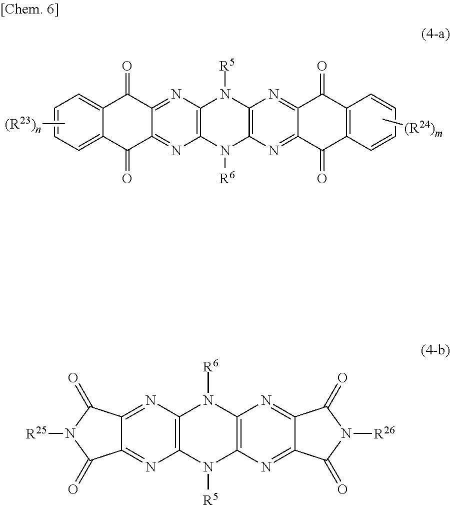

(In the formulae: R²³ to R²⁶ each independently represent a substituted or unsubstituted aryl group having 6 to 60 ring atoms, a substituted or unsubstituted heteroaryl group having 5 to 60 ring atoms, a substituted or unsubstituted alkyl group having 1 to 50 carbon atoms, a substituted or unsubstituted cycloalkyl group having 3 to 50 carbon atoms, or a substituted or unsubstituted haloalkyl group having 1 to 50 carbon atoms; and

n and m each represent an integer of 1 to 4.)

Next, specific examples of the respective groups of the nitrogen-containing heterocyclic derivatives represented by the formulae (1), (2), (3-a), (3-b), (3-c), (3-d), (3-e), (3-f), (3-g), (3-h), (4-a), and (4-b) are described.

Substituents of the substituted or unsubstituted aryl group having 6 to 60 ring atoms and the substituted or unsubstituted heteroaryl group having 5 to 60 ring atoms each represented by any one of R¹ to R²⁶ are, for example, an alkyl group having 1 to 10 carbon atoms, an alkoxy group having 1 to 10 carbon atoms, a hydroxy group, an amino group, a cyano group, a nitro group, and a halogen atom.

The aryl group and the heteroaryl group may each have one or a plurality of the substituents. Specific examples of the groups include a phenyl group, a 1-naphthyl group, a 2-naphthyl group, a 1-anthryl group, a 2-anthryl group, a 9-anthryl group, a 1-phenanthryl group, a 2-phenanthryl group, a 3-phenanthryl group, a 4-phenanthryl group, a 9-phenanthryl group, a 1-naphthacenyl group, a 2-naphthacenyl group, a 9-naphthacenyl group, a 1-pyrenyl group, a 2-pyrenyl group, a 4-pyrenyl group, a 2-biphenylyl group, a 3-biphenylyl group, a 4-biphenylyl group, a p-terphenyl-4-yl group, a p-terphenyl-3-yl group, a p-terphenyl-2-yl group, an m-terphenyl-4-yl group, an m-terphenyl-3-yl group, an m-terphenyl-2-yl group, an o-tolyl group, an m-tolyl group, a p-tolyl group, a p-t-butylphenyl group, a p-(2-phenylpropyl)phenyl group, a 3-methyl-2-naphthyl group, a 4-methyl-1-naphthyl group, a 4-methyl-1-anthryl group, a 4′-methylbiphenylyl group, a 4″-t-butyl-p-terphenyl-4-yl group, a fluoranthenyl group, a fluorenyl group, a 1-pyrrolyl group, a 2-pyrrolyl group, a 3-pyrrolyl group, a pyrazinyl group, a 2-pyridinyl group, a 3-pyridinyl group, a 4-pyridinyl group, a 1-indolyl group, a 2-indolyl group, a 3-indolyl group, a 4-indolyl group, a 5-indolyl group, a 6-indolyl group, a 7-indolyl group, a 1-isoindolyl group, a 2-isoindolyl group, a 3-isoindolyl group, a 4-isoindolyl group, a 5-isoindolyl group, a 6-isoindolyl group, a 7-isoindolyl group, a 2-furyl group, a 3-furyl group, a 2-benzofuranyl group, a 3-benzofuranyl group, a 4-benzofuranyl group, a 5-benzofuranyl group, a 6-benzofuranyl group, a 7-benzofuranyl group, a 1-isobenzofuranyl group, a 3-isobenzofuranyl group, a 4-isobenzofuranyl group, a 5-isobenzofuranyl group, a 6-isobenzofuranyl group, a 7-isobenzofuranyl group, a quinolyl group, a 3-quinolyl group, a 4-quinolyl group, a 5-quinolyl group, a 6-quinolyl group, a 7-quinolyl group, an 8-quinolyl group, a 1-isoquinolyl group, a 3-isoquinolyl group, a 4-isoquinolyl group, a 5-isoquinolyl group, a 6-isoquinolyl group, a 7-isoquinolyl group, an 8-isoquinolyl group, a 2-quinoxalinyl group, a 5-quinoxalinyl group, a 6-quinoxalinyl group, a 1-carbazolyl group, a 2-carbazolyl group, a 3-carbazolyl group, a 4-carbazolyl group, a 9-carbazolyl group, a 1-phenanthridinyl group, a 2-phenanthridinyl group, a 3-phenanthridinyl group, a 4-phenanthridinyl group, a 6-phenanthridinyl group, a 7-phenanthridinyl group, an 8-phenanthridinyl group, a 9-phenanthridinyl group, a 10-phenanthridinyl group, a 1-acridinyl group, a 2-acridinyl group, a 3-acridinyl group, a 4-acridinyl group, a 9-acridinyl group, a 1,7-phenanthrolin-2-yl group, a 1,7-phenanthrolin-3-yl group, a 1,7-phenanthrolin-4-yl group, a 1,7-phenanthrolin-5-yl group, a 1,7-phenanthrolin-6-yl group, a 1,7-phenanthrolin-8-yl group, a 1,7-phenanthrolin-9-yl group, a 1,7-phenanthrolin-10-yl group, a 1,8-phenanthrolin-2-yl group, a 1,8-phenanthrolin-3-yl group, a 1,8-phenanthrolin-4-yl group, a 1,8-phenanthrolin-5-yl group, a 1,8-phenanthrolin-6-yl group, a 1,8-phenanthrolin-7-yl group, a 1,8-phenanthrolin-9-yl group, a 1,8-phenanthrolin-10-yl group, a 1,9-phenanthrolin-2-yl group, a 1,9-phenanthrolin-3-yl group, a 1,9-phenanthrolin-4-yl group, a 1,9-phenanthrolin-5-yl group, a 1,9-phenanthrolin-6-yl group, a 1,9-phenanthrolin-7-yl group, a 1,9-phenanthrolin-8-yl group, a 1,9-phenanthrolin-10-yl group, a 1,10-phenanthrolin-2-yl group, a 1,10-phenanthrolin-3-yl group, a 1,10-phenanthrolin-4-yl group, a 1,10-phenanthrolin-5-yl group, a 2,9-phenanthrolin-1-yl group, a 2,9-phenanthrolin-3-yl group, a 2,9-phenanthrolin-4-yl group, a 2,9-phenanthrolin-5-yl group, a 2,9-phenanthrolin-6-yl group, a 2,9-phenanthrolin-7-yl group, a 2,9-phenanthrolin-8-yl group, a 2,9-phenanthrolin-10-yl group, a 2,8-phenanthrolin-1-yl group, a 2,8-phenanthrolin-3-yl group, a 2,8-phenanthrolin-4-yl group, a 2,8-phenanthrolin-5-yl group, a 2,8-phenanthrolin-6-yl group, a 2,8-phenanthrolin-7-yl group, a 2,8-phenanthrolin-9-yl group, a 2,8-phenanthrolin-10-yl group, a 2,7-phenanthrolin-1-yl group, a 2,7-phenanthrolin-3-yl group, a 2,7-phenanthrolin-4-yl group, a 2,7-phenanthrolin-5-yl group, a 2,7-phenanthrolin-6-yl group, a 2,7-phenanthrolin-8-yl group, a 2,7-phenanthrolin-9-yl group, a 2,7-phenanthrolin-10-yl group, a 1-phenazinyl group, a 2-phenazinyl group, a 1-phenothiazinyl group, a 2-phenothiazinyl group, a 3-phenothiazinyl group, a 4-phenothiazinyl group, a 10-phenothiazinyl group, a 1-phenoxazinyl group, a 2-phenoxazinyl group, a 3-phenoxazinyl group, a 4-phenoxazinyl group, a 10-phenoxazinyl group, a 2-oxazolyl group, a 4-oxazolyl group, a 5-oxazolyl group, a 2-oxadiazolyl group, a 5-oxadiazolyl group, a 3-furazanyl group, a 2-thienyl group, a 3-thienyl group, a 2-methylpyrrol-1-yl group, a 2-methylpyrrol-3-yl group, a 2-methylpyrrol-4-yl group, a 2-methylpyrrol-5-yl group, a 3-methylpyrrol-1-yl group, a 3-methylpyrrol-2-yl group, a 3-methylpyrrol-4-yl group, a 3-methylpyrrol-5-yl group, a 2-t-butylpyrrol-4-yl group, a 3-(2-phenylpropyl)pyrrol-1-yl group, a 2-methyl-1-indolyl group, a 4-methyl-1-indolyl group, a 2-methyl-3-indolyl group, a 4-methyl-3-indolyl group, a 2-t-butyl-1-indolyl group, a 4-t-butyl-1-indolyl group, a 2-t-butyl-3-indolyl group, and a 4-t-butyl-3-indolyl group.

Of those, a substituted or unsubstituted aryl group having 6 to 30 ring atoms, and a substituted or unsubstituted heteroaryl group having 5 to 30 ring atoms are preferred, and a substituted or unsubstituted aryl group having 6 to 18 ring atoms, and a substituted or unsubstituted heteroaryl group having 5 to 18 ring atoms are more preferred. Examples of such groups include a phenyl group, a naphthyl group, a biphenylyl group, an anthranyl group, a phenanthryl group, a pyrenyl group, a chrysenyl group, a fluoranthenyl group, a fluorenyl group, a pyridinyl group, a quinolyl group, an isoquinolyl group, and a phenanthryl group. Those groups may each be substituted with any one of the above-mentioned substituents.

The substituted or unsubstituted alkyl group having 1 to 50 carbon atoms, and the substituted or unsubstituted haloalkyl group having 1 to 50 carbon atoms each represented by any one of R¹ to R²⁶ may be linear or branched. In addition, a substituent for any such group is, for example, a hydroxy group, an amino group, a cyano group, a nitro group, or a halogen atom.

The alkyl group and the haloalkyl group may each have one or a plurality of the substituents. Specific examples of the groups include a methyl group, an ethyl group, a propyl group, an isopropyl group, an n-butyl group, an s-butyl group, an isobutyl group, a t-butyl group, an n-pentyl group, an n-hexyl group, an n-heptyl group, an n-octyl group, fluorine-substituted alkyl groups each having 1 to 50 carbon atoms such as a trifluoromethyl group and a trifluoroethyl group, a hydroxymethyl group, a 1-hydroxyethyl group, a 2-hydroxyethyl group, a 2-hydroxyisobutyl group, a 1,2-dihydroxyethyl group, a 1,3-dihydroxyisopropyl group, a 2,3-dihydroxy-t-butyl group, a 1,2,3-trihydroxypropyl group, substituted or unsubstituted haloalkyl groups each having 1 to 50 carbon atoms such as a chloromethyl group, a 1-chloroethyl group, a 2-chloroethyl group, a 2-chloroisobutyl group, a 1,2-dichloroethyl group, a 1,3-dichloroisopropyl group, a 2,3-dichloro-t-butyl group, a 1,2,3-trichloropropyl group, a bromomethyl group, a 1-bromoethyl group, a 2-bromoethyl group, a 2-bromoisobutyl group, a 1,2-dibromoethyl group, a 1,3-dibromoisopropyl group, a 2,3-dibromo-t-butyl group, a 1,2,3-tribromopropyl group, an iodomethyl group, a 1-iodoethyl group, a 2-iodoethyl group, a 2-iodoisobutyl group, a 1,2-diiodoethyl group, a 1,3-diiodoisopropyl group, a 2,3-diiodo-t-butyl group, and a 1,2,3-triiodopropyl group, an aminomethyl group, a 1-aminoethyl group, a 2-aminoethyl group, a 2-aminoisobutyl group, a 1,2-diaminoethyl group, a 1,3-diaminoisopropyl group, a 2,3-diamino-t-butyl group, a 1,2,3-triaminopropyl group, a cyanomethyl group, a 1-cyanoethyl group, a 2-cyanoethyl group, a 2-cyanoisobutyl group, a 1,2-dicyanoethyl group, a 1,3-dicyanoisopropyl group, a 2,3-dicyano-t-butyl group, a 1,2,3-tricyanopropyl group, a nitromethyl group, a 1-nitroethyl group, a 2-nitroethyl group, a 2-nitroisobutyl group, a 1,2-dinitroethyl group, a 1,3-dinitroisopropyl group, a 2,3-dinitro-t-butyl group, and a 1,2,3-trinitropropyl group.

Of those, a substituted or unsubstituted alkyl group having 1 to 28 carbon atoms, or a haloalkyl group having 1 to 28 carbon atoms is preferred, and a substituted or unsubstituted alkyl group having 1 to 6 carbon atoms, or a fluorine-substituted alkyl group having 1 to 6 carbon atoms is more preferred. Examples of such groups include a methyl group, an ethyl group, a butyl group, a pentyl group, a hexyl group, a trifluoromethyl group, and a trifluoroethyl group.

The substituted or unsubstituted cycloalkyl group having 3 to 50 carbon atoms represented by any one of R¹ to R²⁶ may be monocyclic or polycyclic. In addition, a substituent for the group is, for example, an alkyl group having 1 to 10 carbon atoms, an alkoxy group having 1 to 10 carbon atoms, a hydroxy group, an amino group, a cyano group, a nitro group, or a halogen atom. The cycloalkyl group may have one or a plurality of the substituents. Specific examples of the cycloalkyl group include a cyclopropyl group, a cyclobutyl group, a cyclopentyl group, a cyclohexyl group, a 4-methylcyclohexyl group, a 1-adamantyl group, a 2-adamantyl group, a 1-norbornyl group, and a 2-norbornyl group.

Of those, a substituted or unsubstituted cycloalkyl group having 3 to 20 carbon atoms is preferred, and a substituted or unsubstituted cycloalkyl group having 3 to 9 carbon atoms is more preferred. Examples of such group include a cyclopropyl group, a cyclobutyl group, a cyclopentyl group, a cyclohexyl group, and a methylcyclohexyl group.

Examples of the respective groups each represented by R′ include those obtained by making the above-mentioned specific examples described for R¹ to R²⁶ divalent.

Groups except those described above each represented by any one of R¹ to R⁴ are, for example, the following groups.

The substituted or unsubstituted aryloxycarbonyl group having 6 to 60 ring atoms is a group represented by —COOY, and examples of Y and the substituent include the same examples as those described for the aryl group.

The substituted or unsubstituted alkoxycarbonyl group having 1 to 50 carbon atoms is a group represented by —COOZ, and examples of Z and the substituent include the same examples as those described for the alkyl group.

The substituted or unsubstituted arylcarbonyl group having 6 to 60 ring atoms is a group represented by —COY, and examples of Y and the substituent include the same examples as those described for the aryl group.

The substituted or unsubstituted alkylcarbonyl group having 1 to 50 carbon atoms is a group represented by —COZ, and examples of Z and the substituent include the same examples as those described for the alkyl group.

The substituted or unsubstituted arylsulfonyl group having 6 to 60 ring atoms is a group represented by —SO₂Y, and examples of Y and the substituent include the same examples as those described for the aryl group.

The substituted or unsubstituted alkylsulfonyl group having 1 to 50 carbon atoms is a group represented by —SO₂Z, and examples of Z and the substituent include the same examples as those described for the alkyl group.

The substituted or unsubstituted arylsulfinyl group having 6 to 60 ring atoms is a group represented by —SOY, and examples of Y and the substituent include the same examples as those described for the aryl group.

The substituted or unsubstituted alkylsulfinyl group having 1 to 50 carbon atoms is a group represented by —SOZ, and examples of Z and the substituent include the same examples as those described for the alkyl group.

The substituted or unsubstituted carbamoyl group is a group represented by —CONY″₂, and examples of Y″ and the substituent include the same examples as those described for the alkyl group and aryl group. In addition, Y″ may represent a hydrogen atom.

Examples of the halogen atom include a fluorine atom, a chlorine atom, a bromine atom, and an iodine atom.

The other groups are, for example, a cyano group and a nitro group.

Of those, a cyano group, a nitro group, an arylcarbonyl group having 6 to 30 ring atoms, an alkylcarbonyl group having 1 to 28 carbon atoms, a dialkylcarbamoyl group having 1 to 28 carbon atoms, a diarylcarbamoyl group, an aryloxycarbonyl group having 6 to 30 ring atoms, or an alkoxycarbonyl group having 1 to 28 carbon atoms is preferred, and a cyano group, an arylcarbonyl group having 6 to 18 ring atoms, an alkylcarbonyl group having 1 to 6 carbon atoms, a dialkylcarbamoyl group having 1 to 6 carbon atoms, a diarylcarbamoyl group having 6 to 18 ring atoms, an aryloxycarbonyl group having 6 to 18 ring atoms, an alkoxycarbonyl group having 1 to 6 carbon atoms, or the like is more preferred.

Examples of the ring structure which adjacent groups of R¹ to R⁴ in the formula (1) may be bonded to each other to form include aromatic hydrocarbon rings such as a benzene ring, heterocyclic rings such as a pyridine ring, a pyrimidine ring, a triazine ring, a pyrazine ring, a furan ring, a pyrrole ring, a thiophene ring, an imidazole ring, an oxazole ring, and a thiazole ring, and such pyrrolidinedione rings, dihydrofurandione rings, cyclohexanedione rings, and dihydronaphthalenedione rings as represented by the formulae (3-c), (4-a), and (4-b).

At least one of R¹ to R⁴ in the formula (1) preferably represents an electron-withdrawing substituent. The term “electron-withdrawing substituent” as used herein refers to a substituted or unsubstituted haloalkyl group having 1 to 50 carbon atoms, a substituted or unsubstituted aryloxycarbonyl group having 6 to 60 ring atoms, a substituted or unsubstituted alkoxycarbonyl group having 1 to 50 carbon atoms, a substituted or unsubstituted carbamoyl group, a substituted or unsubstituted arylcarbonyl group having 6 to 60 ring atoms, a substituted or unsubstituted alkylcarbonyl group having 1 to 50 carbon atoms, a substituted or unsubstituted arylsulfonyl group having 6 to 60 ring atoms, a substituted or unsubstituted alkylsulfonyl group having 1 to 50 carbon atoms, a substituted or unsubstituted arylsulfinyl group having 6 to 60 ring atoms, a substituted or unsubstituted alkylsulfinyl group having 1 to 50 carbon atoms, a cyano group, or a nitro group.

Of those, a cyano group, a haloalkyl group having 1 to 28 carbon atoms, a nitro group, an arylcarbonyl group having 6 to 30 ring atoms, an alkylcarbonyl group having 1 to 28 carbon atoms, a dialkylcarbamoyl group having 1 to 28 carbon atoms, a diarylcarbamoyl group, an aryloxycarbonyl group having 6 to 30 ring atoms, or an alkoxycarbonyl group having 1 to 28 carbon atoms is preferred, and a cyano group, a fluorine-substituted alkyl group having 1 to 6 carbon atoms, an arylcarbonyl group having 6 to 18 ring atoms, an alkylcarbonyl group having 1 to 6 carbon atoms, a dialkylcarbamoyl group having 1 to 6 carbon atoms, a diarylcarbamoyl group having 6 to 18 ring atoms, an aryloxycarbonyl group having 6 to 18 ring atoms, an alkoxycarbonyl group having 1 to 6 carbon atoms, or the like is more preferred.

Exemplary compounds for the nitrogen-containing heterocyclic derivative represented by the formula (1) of the present invention are shown below, but the derivative is not limited thereto.

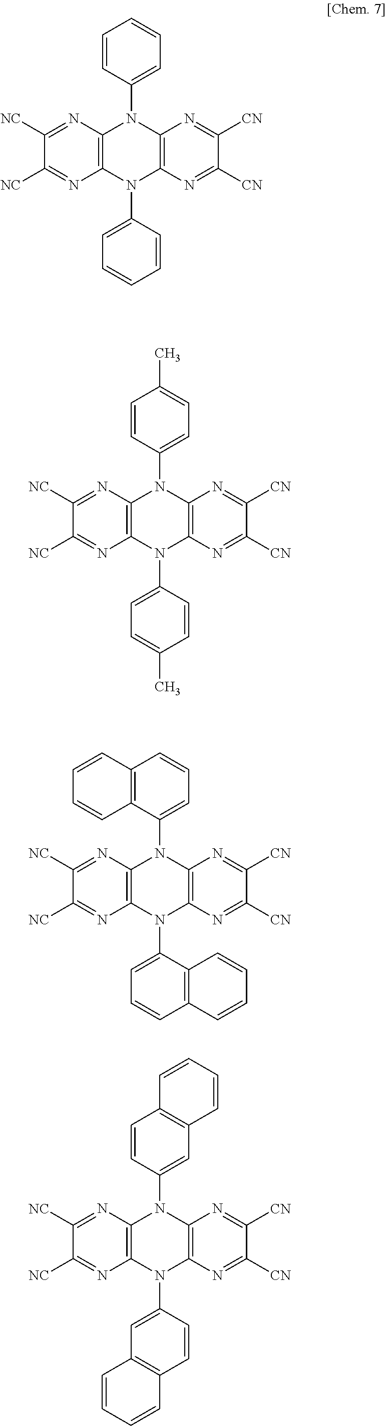

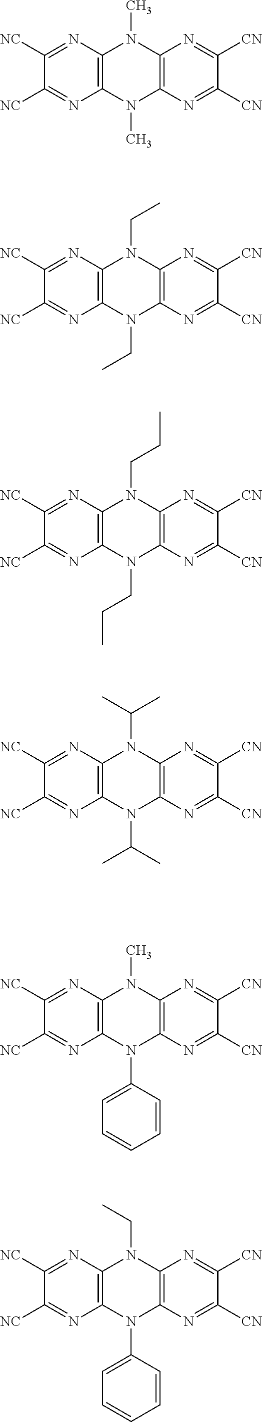

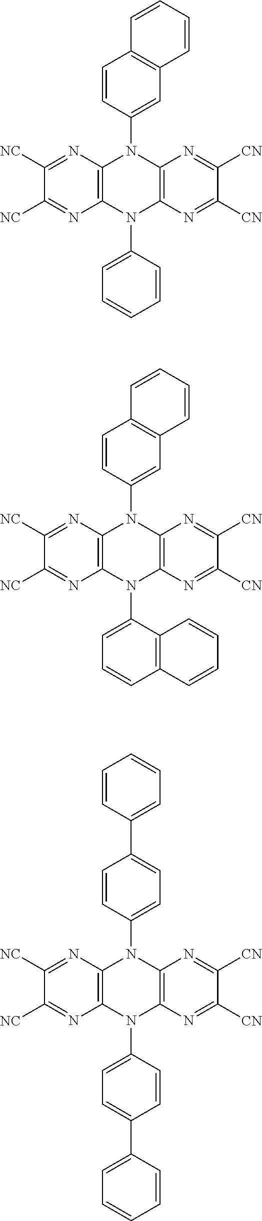

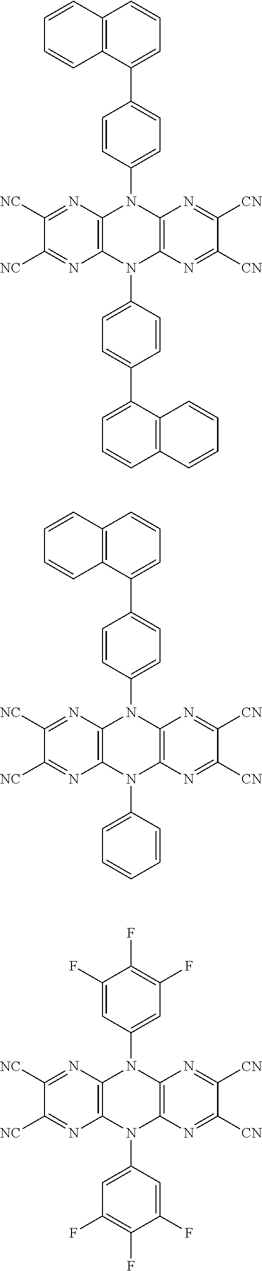

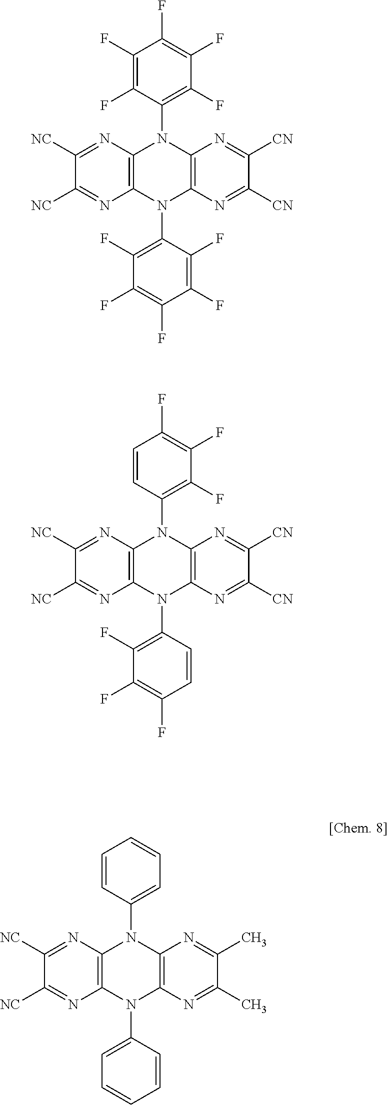

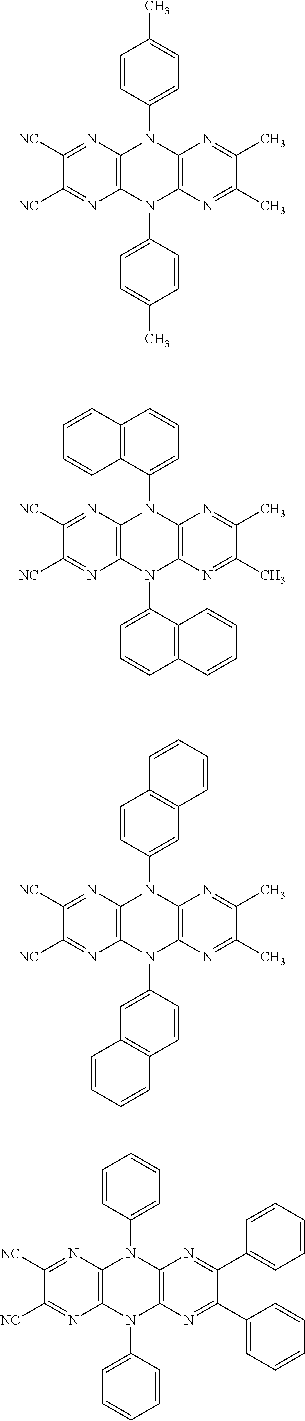

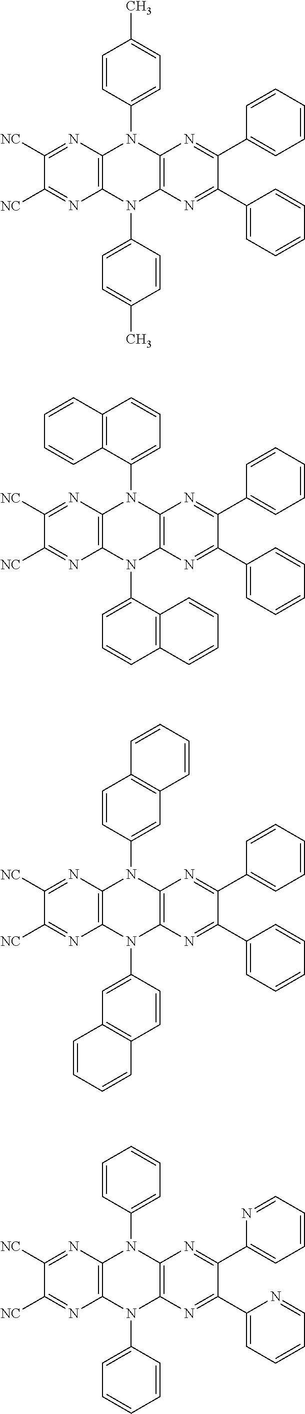

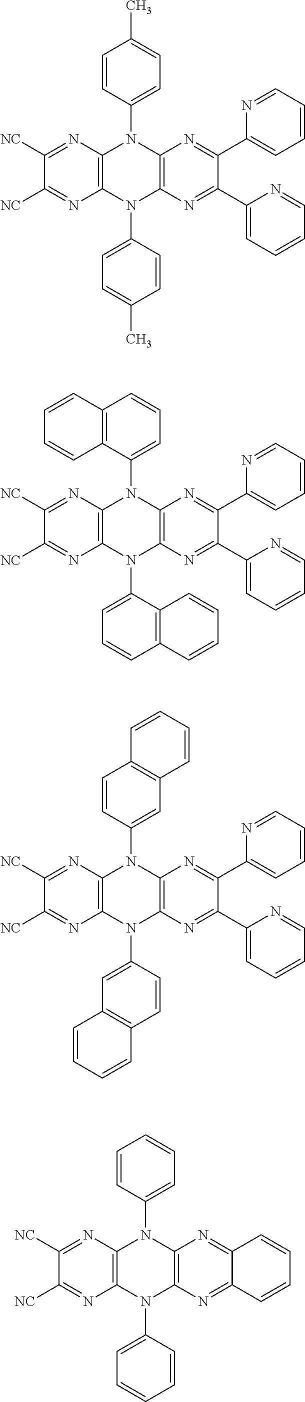

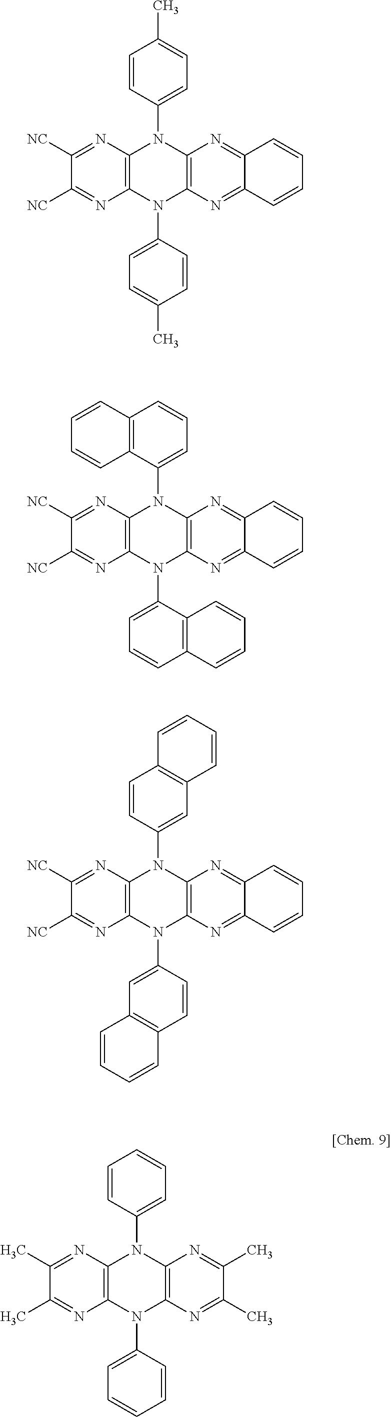

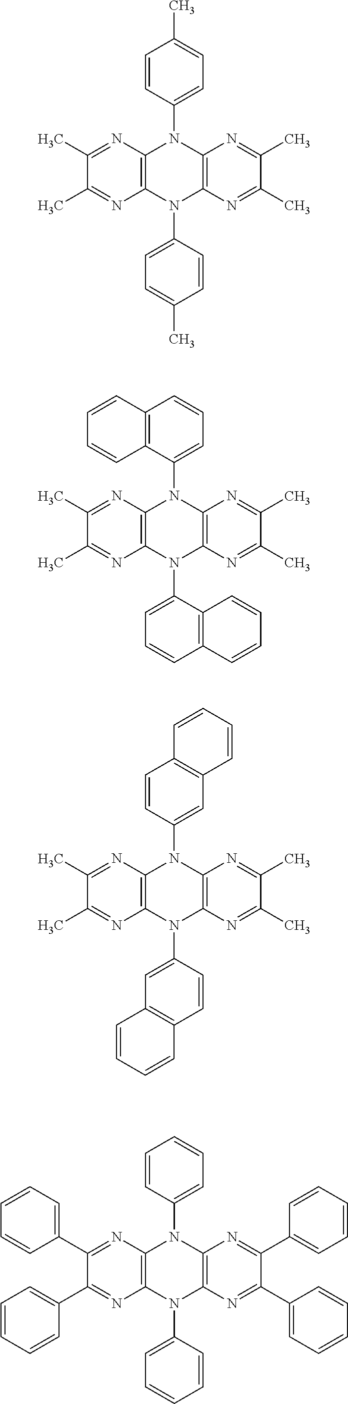

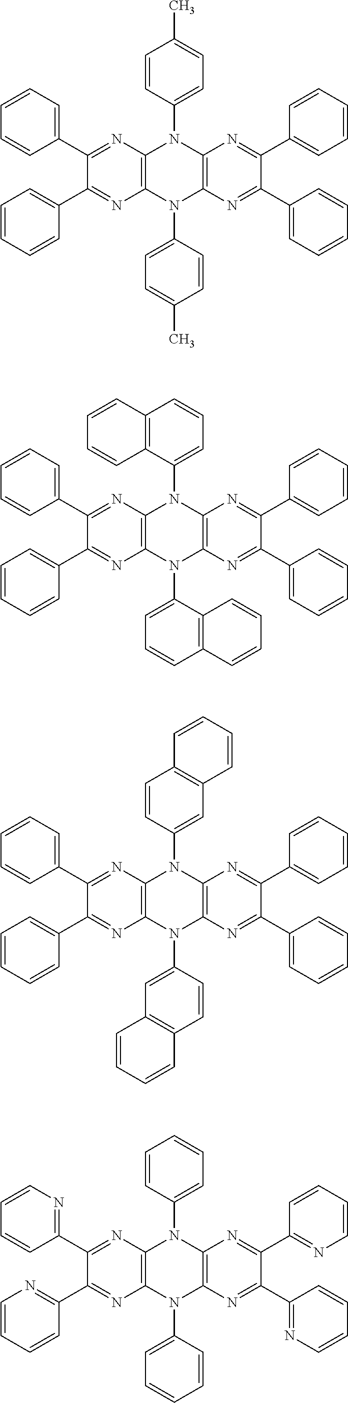

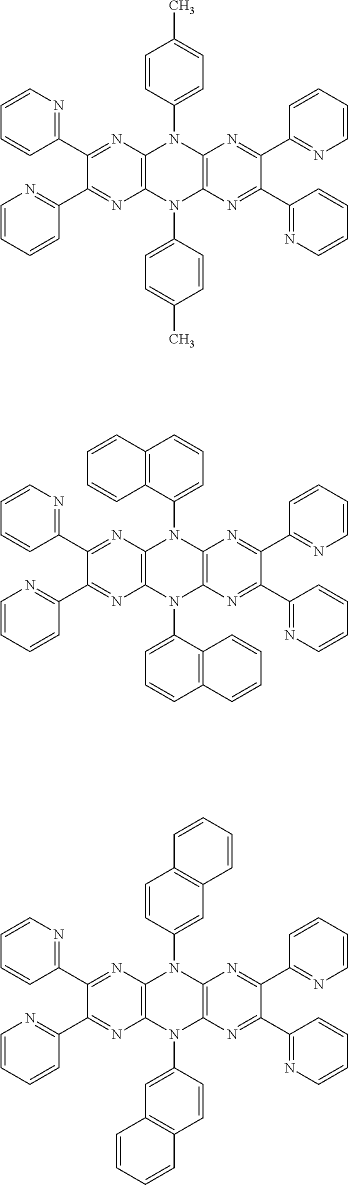

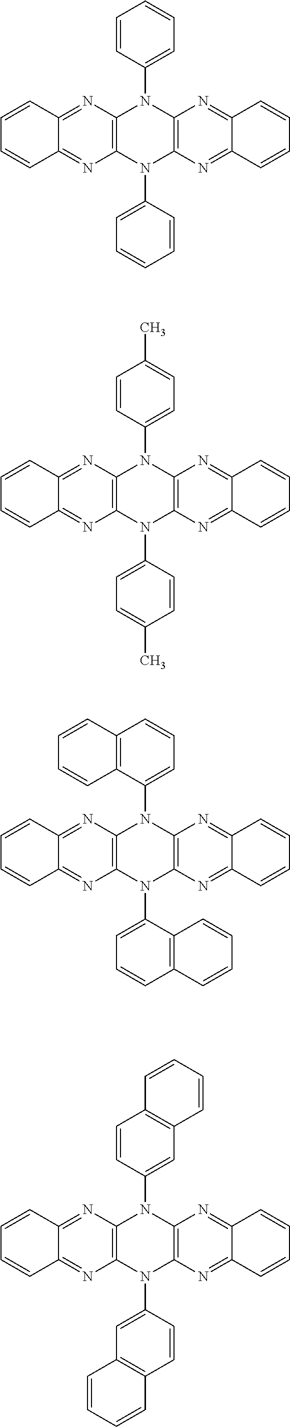

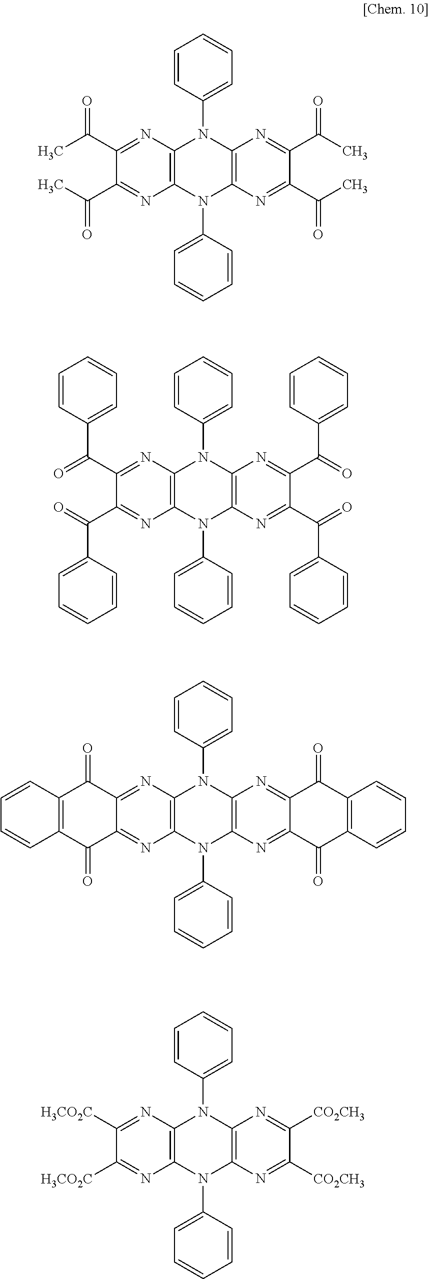

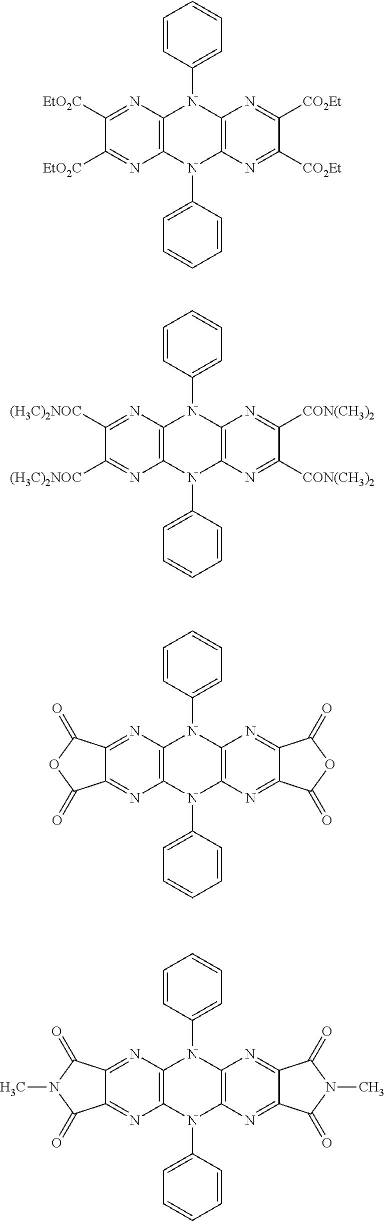

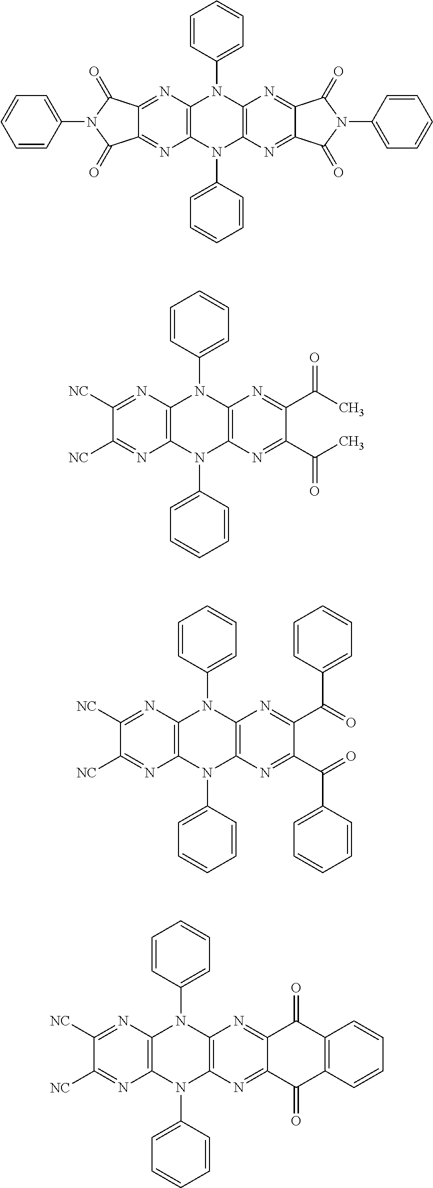

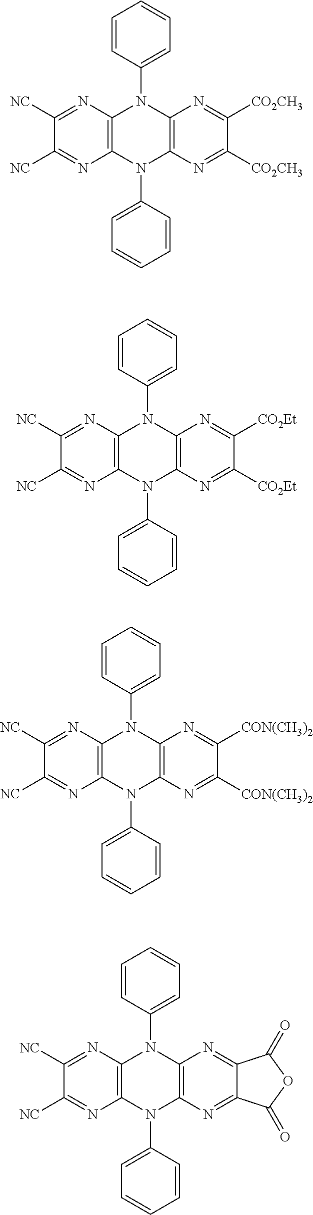

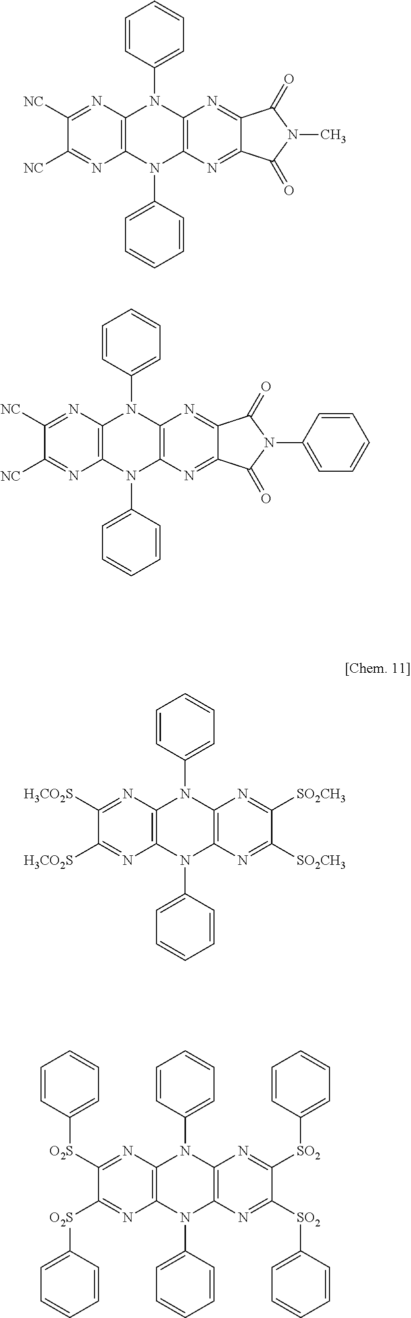

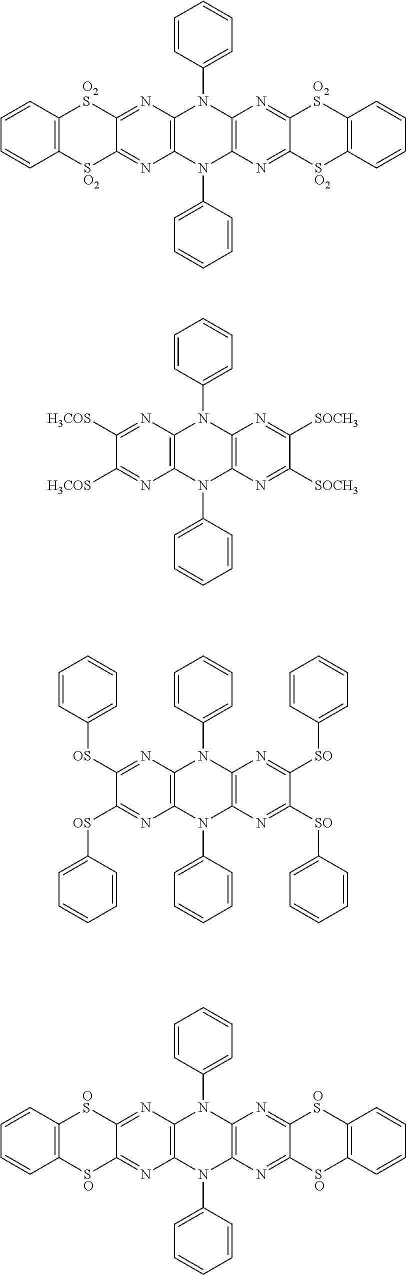

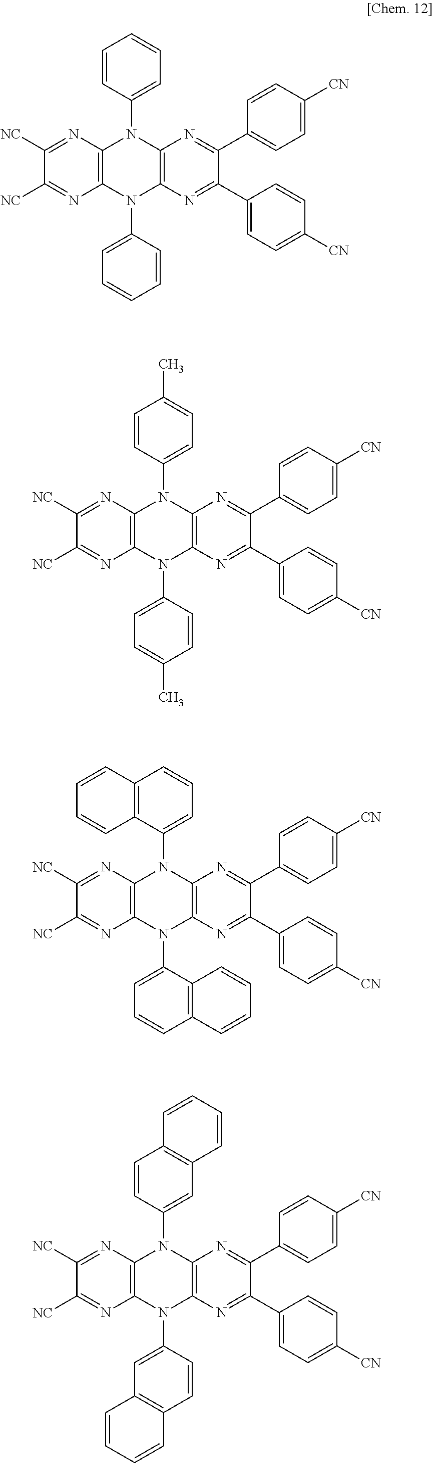

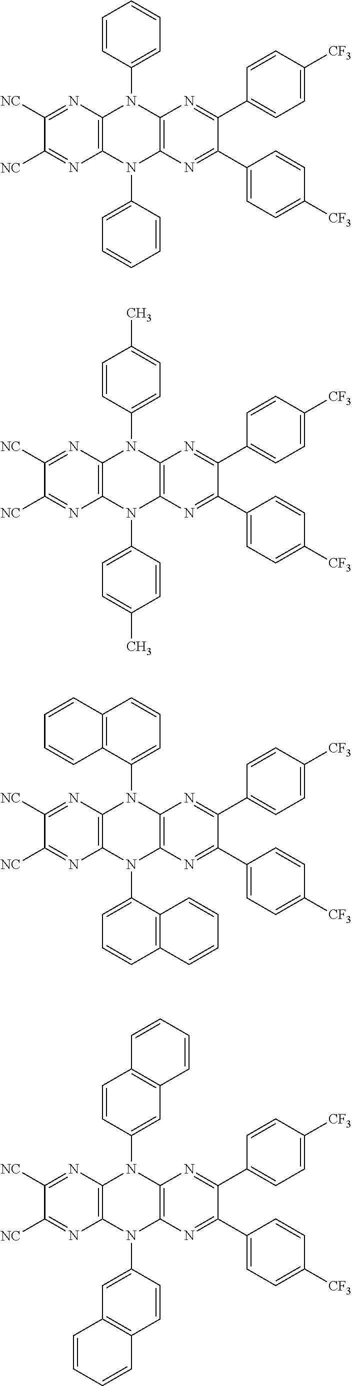

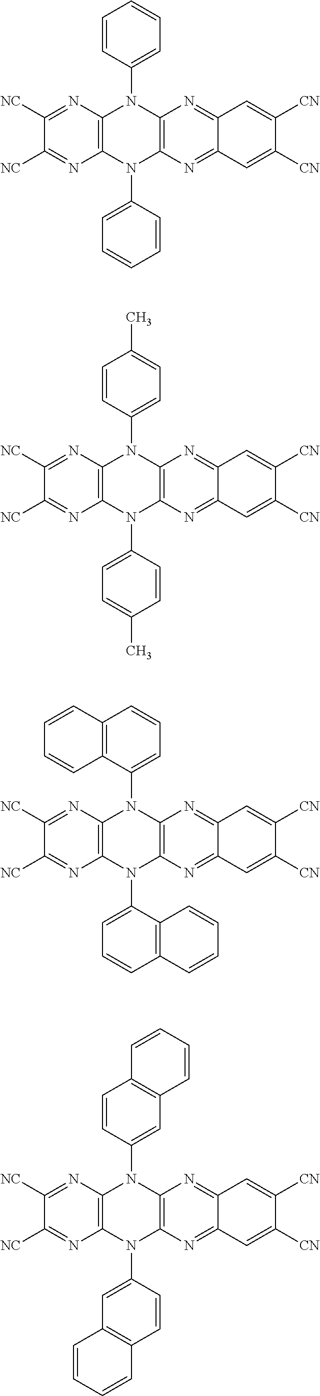

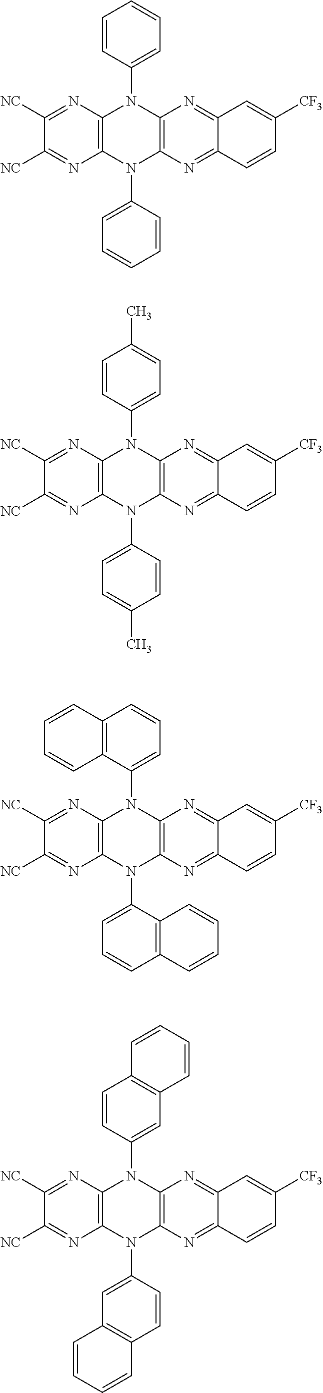

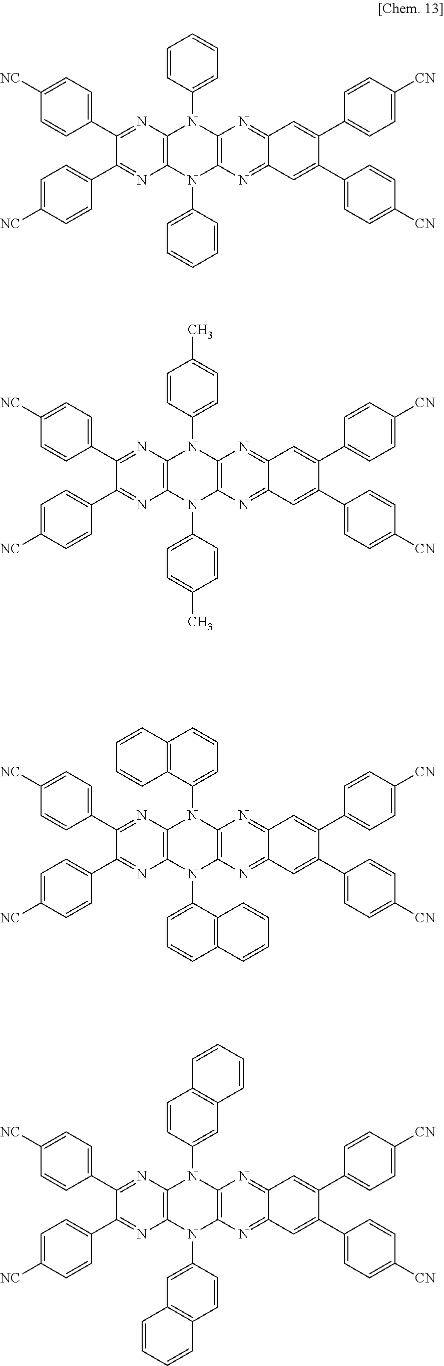

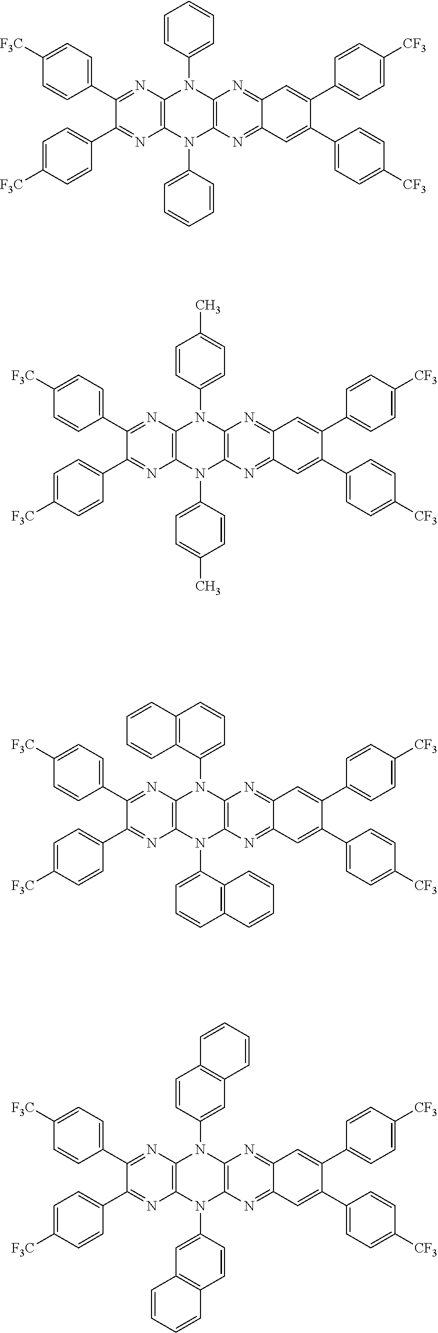

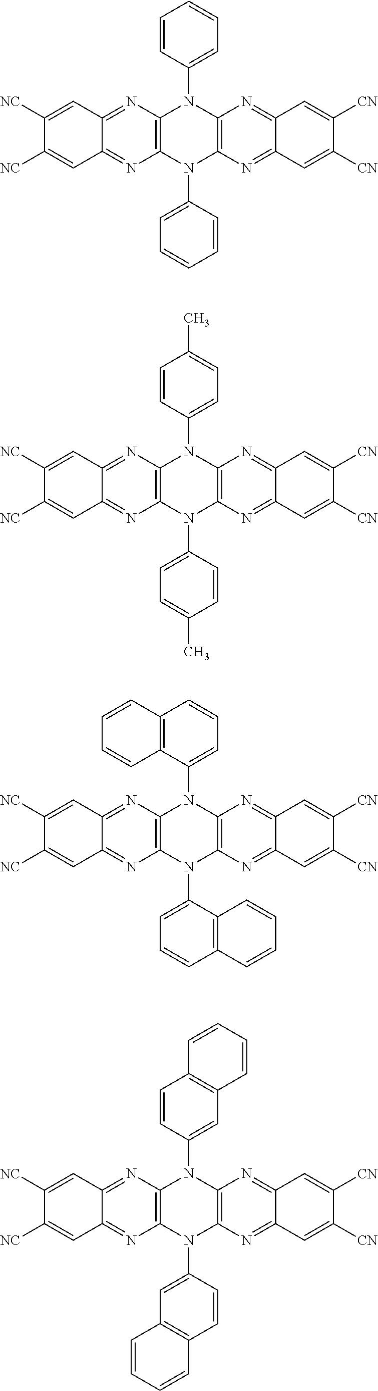

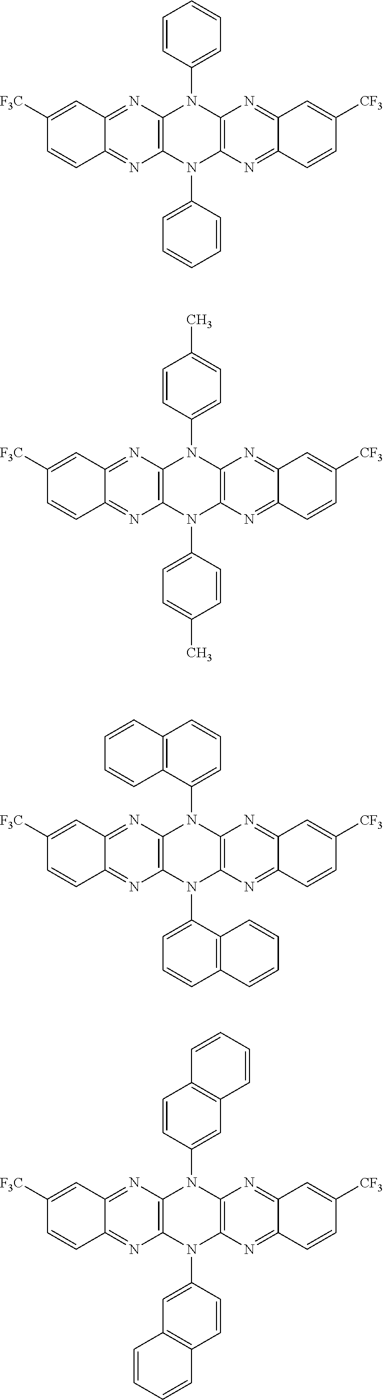

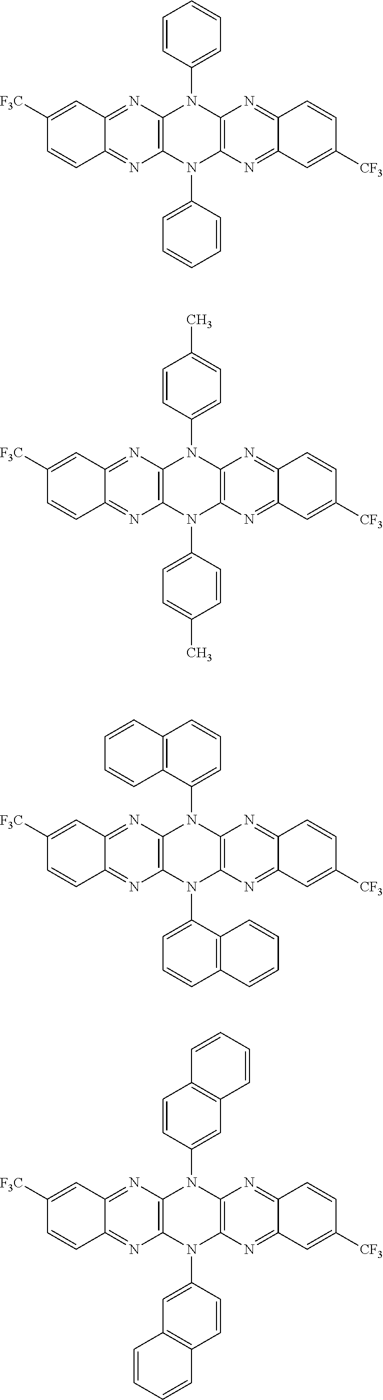

### (Method of Synthesizing Nitrogen-Containing Heterocyclic Derivative)

The nitrogen-containing heterocyclic derivative of the present invention can be synthesized by, for example, any one of the following methods:

(a) to heat an intermediate represented by the following formula (I) under a nitrogen atmosphere (e.g., J. Heterocyclic. Chem., vol. 34, p. 653, 1997);

(b) to similarly heat intermediates represented by the formulae (II) and (III); and

(c) to introduce a necessary substituent into an intermediate represented by the formula (IV).

The resultant compound may be further purified by, for example, being recrystallized from a proper solution.

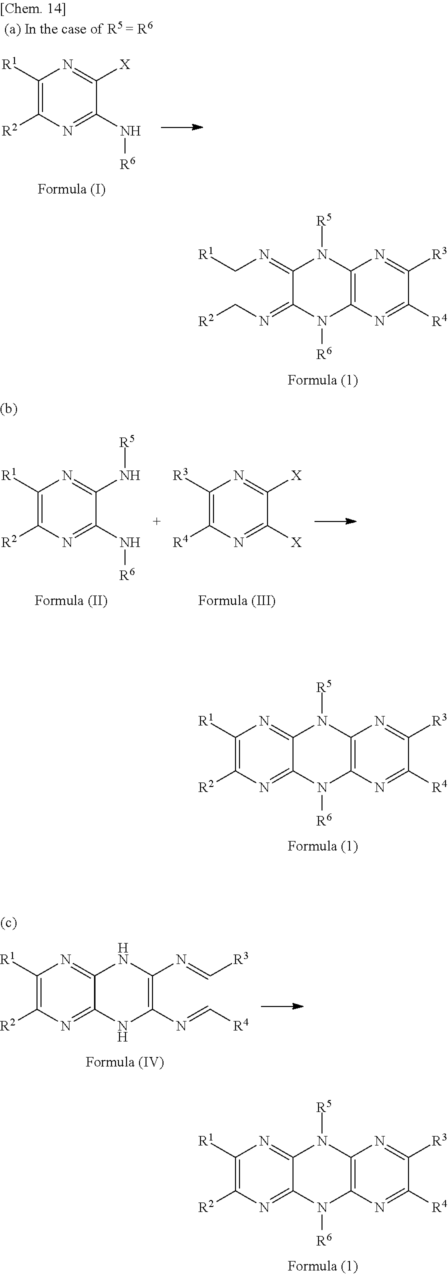

Next, an organic EL device using the nitrogen-containing heterocyclic derivative of the present invention is described.

The organic EL device of the present invention is an organic electroluminescence device having one or a plurality of organic layers interposed between a cathode and an anode, and at least one layer of the organic layers contains the nitrogen-containing heterocyclic derivative of the present invention.

Representative device configurations for the organic EL device of the present invention include, but not limited to, the following configurations:

(1) an anode/light emitting layer/cathode;

(2) an anode/hole injecting layer/light emitting layer/cathode;

(3) an anode/light emitting layer/electron injecting layer/cathode;

(4) an anode/light emitting layer/electron transporting layer/electron injecting layer/cathode;

(5) an anode/hole injecting layer/light emitting layer/electron injecting layer/cathode;

(6) an anode/hole injecting layer/light emitting layer/electron transporting layer/electron injecting layer/cathode;

(7) an anode/organic semiconductor layer/light emitting layer/cathode;

(8) an anode/organic semiconductor layer/electron barrier layer/light emitting layer/cathode;

(9) an anode/organic semiconductor layer/light emitting layer/adhesion improving layer/cathode;

(10) an anode/hole injecting layer/hole transporting layer/light emitting layer/electron injecting layer/cathode;

(11) an anode/hole injecting layer/hole transporting layer/light emitting layer/electron transporting layer/electron injecting layer/cathode;

(12) an anode/insulating layer/light emitting layer/insulating layer/cathode;

(13) an anode/inorganic semiconductor layer/insulating layer/light emitting layer/insulating layer/cathode;

(14) an anode/organic semiconductor layer/insulating layer/light emitting layer/insulating layer/cathode;

(15) an anode/insulating layer/hole injecting layer/hole transporting layer/light emitting layer/insulating layer/cathode;

(16) an anode/insulating layer/hole injecting layer/hole transporting layer/light emitting layer/electron injecting layer/cathode; and

(17) an anode/insulating layer/hole injecting layer/hole transporting layer/light emitting layer/electron transporting layer/electron injecting layer/cathode.

Of those, the configuration (10) or (11) is preferred.

The nitrogen-containing heterocyclic derivative of the present invention may be used in any organic layer in the above-mentioned organic EL device. For example, the nitrogen-containing heterocyclic derivative of the present invention may be incorporated as a light emitting material for an organic EL device into the light emitting layer by taking advantage of its light emitting property. Alternatively, the derivative may be incorporated as a hole injecting material or hole transporting material for an organic EL device into the hole injecting layer or the hole transporting layer by taking advantage of its hole injecting or transporting property. Further, the derivative may be incorporated as an electron injecting material or electron transporting material for an organic EL device into the electron injecting layer or the electron transporting layer by taking advantage of its electron injecting or transporting property.

(Light-Transmissive Substrate)

The organic EL device of the present invention is produced on a light-transmissive substrate in the case of such a bottom surface emission-type or bottom emission-type organic EL device that emitted light exits from the substrate side. The light-transmissive substrate is preferably a substrate which supports the organic EL device, has a transmittance of light of 50% or more in the visible region of 400 to 700 nm, and is flat and smooth. Examples of the light-transmissive substrate include glass plates and polymer plates. Specific examples of the glass plates include plates formed of soda-lime glass, glass containing barium and strontium, lead glass, aluminosilicate glass, borosilicate glass, barium borosilicate glass, and quartz. In addition, specific examples of the polymer plates include plates formed of polycarbonate, acrylic, polyethylene terephthalate, polyether sulfide, and polysulfone. Alternatively, the substrate may be a TFT substrate on which a TFT for driving is formed.

In addition, in the case of such a top surface emission-type or top emission-type organic EL device that emitted light exits from the upper portion of the device, a light reflecting layer made of a proper metal such as aluminum is required to be provided on the above-mentioned substrate.

(Anode)

The anode of the organic EL device of the present invention has the function of injecting holes into the hole transporting layer or the light emitting layer. It is effective that the anode has a work function of 4.5 eV or more. Specific examples of the material for the anode to be used in the present invention include indium tin oxide (ITO) alloys, tin oxide (NESA), indium zinc oxide (IZO), gold, silver, platinum, and copper.

The anode can be prepared by forming a thin film of the electrode material described above in accordance with a process such as a vapor deposition process or a sputtering process.

In the case of a bottom surface emission-type or bottom emission-type organic EL device, it is preferred that the anode have a transmittance of the emitted light greater than 10%. It is also preferred that the sheet resistivity of the anode be several hundred Ω/□ or less. The thickness of the anode is, in general, selected in the range of 10 nm to 1 μm, preferably in the range of 10 to 200 nm although the preferred range may be different depending on the used material.

(Light Emitting Layer)

The light emitting layer of the organic EL device has a combination of the following functions (1) to (3).

(1) Injecting function; the function of injecting holes from the anode or the hole injecting layer and injecting electrons from the cathode or the electron injecting layer when an electric field is applied.

(2) Transporting function: the function of transporting injected charges (i.e., electrons and holes) by the force of the electric field.

(3) Light emitting function: the function of providing a field for recombination of electrons and holes and leading the recombination to the emission of light.

It should be noted that the easiness of injection may be different between holes and electrons and the ability of transportation expressed by the mobility may be different between holes and electrons. It is preferred that any one of the charges be transferred.

A known method such as a vapor deposition method, a spin coating method, or an LB method is applicable to the formation of the light emitting layer. The light emitting layer is particularly preferably a molecular deposit film. The term “molecular deposit film” as used herein refers to a thin film formed by the deposition of a material compound in a vapor phase state, or a film formed by the solidification of a material compound in a solution state or a liquid phase state. The molecular deposit film can be typically distinguished from a thin film formed by the LB method (molecular accumulation film) on the basis of differences between the films in aggregation structure and higher order structure, and functional differences between the films caused by the foregoing differences.

In addition, as disclosed in JP 57-51781 A, the light emitting layer can also be formed by: dissolving a binder such as a resin and a material compound in a solvent to prepare a solution; and forming a thin film from the prepared solution by the spin coating method or the like.

In the organic EL device of the present invention, the light emitting layer may be formed from a light emitting material for an organic EL device containing the nitrogen-containing heterocyclic derivative of the present invention. Further, when desired, the light emitting layer may contain another known light emitting material in addition to the nitrogen-containing heterocyclic derivative of the present invention, or a light emitting layer containing another known light emitting material may also be laminated to the light emitting layer produced from the nitrogen-containing heterocyclic derivative of the present invention as long as the object of the present invention is not adversely affected.

Further, in the organic EL device of the present invention, in addition to the nitrogen-containing heterocyclic derivative of the present invention, the light emitting layer preferably contains an aryl amine compound and/or a styrylamine compound. In this case, the content of the nitrogen-containing heterocyclic derivative of the present invention is preferably in the range of 0.1 to 20 mass %, more preferably in the range of 0.1 to 10 mass %.

Examples of the arylamine compound include compounds each represented by the following formula (A).

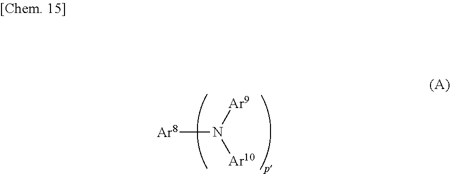

In the formula: Ar⁸ represents a group selected from phenyl, biphenyl, terphenyl, stilbene, and distyrylaryl groups; Ar₉ and Ar₁₀ each represent a hydrogen atom or an aromatic group having 6 to 20 carbon atoms; the aromatic group may be substituted; p′ represents an integer of 1 to 4; and Ar⁹ and/or Ar¹⁰ are/is more preferably substituted with styryl groups/a styryl group.

Here, the aromatic group having 6 to 20 carbon atoms is preferably a phenyl group, a naphthyl group, an anthranyl group, a phenanthryl group, a terphenyl group, or the like.

In addition, examples of the styrylamine compound include compounds each represented by the following formula (B).

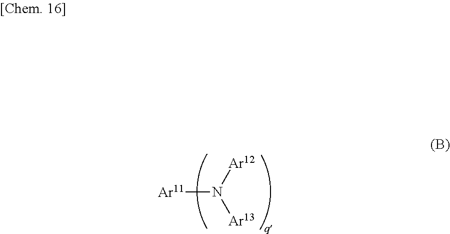

In the formula: Ar¹¹ to Ar¹³ each represent an aryl group which has 6 to 40 ring carbon atoms and which may be substituted; and q′ represents an integer of 1 to 4.

Here, examples of the aryl group having 6 to 40 ring atoms preferably include phenyl, naphthyl, anthranyl, phenanthryl, pyrenyl, coronyl, biphenyl, terphenyl, pyrrolyl, furanyl, thiophenyl, benzothiophenyl, oxadiazolyl, diphenylanthranyl, indolyl, carbazolyl, pyridyl, benzoquinolyl, fluoranthenyl, acenaphthofluoranthenyl, and stilbene. In addition, the aryl group having 5 to 40 ring atoms may further be substituted with a substituent. Examples of the substituent preferably include an alkyl group having 1 to 6 carbon atoms (such as an ethyl group, a methyl group, an isopropyl group, an n-propyl group, an s-butyl group, a t-butyl group, a pentyl group, a hexyl group, a cyclopentyl group, or a cyclohexyl group), an alkoxy group having 1 to 6 carbon atoms (such as an ethoxy group, a methoxy group, an isopropoxy group, an n-propoxy group, an s-butoxy group, a t-butoxy group, a pentoxy group, a hexyloxy group, a cyclopentoxy group, or a cyclohexyloxy group), an aryl group having 6 to 40 ring atoms, an amino group substituted with an aryl group having 6 to 40 ring atoms, an ester group having an aryl group having 6 to 40 ring atoms, an ester group having an alkyl group having 1 to 6 carbon atoms, a cyano group, a nitro group, and a halogen atom (such as chlorine, bromine, or iodine).

Examples of the another known light emitting material include, but are not limited to, anthracene, naphthalene, phenanthrene, pyrene, tetracene, coronene, chrysene, fluorescein, perylene, phthaloperylene, naphthaloperylene, perinone, phthaloperinone, naphthaloperinone, diphenylbutadiene, tetraphenylbutadiene, coumarin, oxadiazole, aldazine, bisbenzoxazoline, bisstyryl, pyrazine, cyclopentadiene, quinoline metal complexes, aminoquinoline metal complexes, benzoquinoline metal complexes, imines, diphenylethylene, vinylanthracene, diaminocarbazole, pyrane, thiopyrane, polymethine, merocyanine, imidazole-chelated oxynoid compounds, quinacridone, rubrene, and fluorescent dyes.

In the light emitting layer of the organic EL device of the present invention, the nitrogen-containing heterocyclic derivative of the present invention can, or the nitrogen-containing heterocyclic derivative and a compound represented by any one of the following formulae (i) to (ix) can each, be used as a host material, and the another known light emitting material can be used as a dopant. In this case, light emitted from the organic EL device has a wavelength specific to the other known light emitting material:

an asymmetric anthracene represented by the following formula (1);

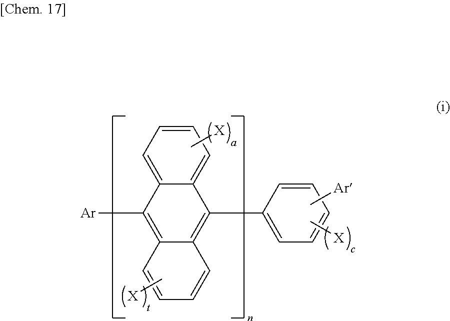

(In the formula: Ar represents a substituted or unsubstituted fused aromatic group having 10 to 50 ring carbon atoms;

Ar′ represents a substituted or unsubstituted aromatic group having 6 to 50 ring carbon atoms;

X represents a substituted or unsubstituted aromatic group having 6 to 50 ring carbon atoms, a substituted or unsubstituted aromatic heterocyclic group having 5 to 50 ring atoms, a substituted or unsubstituted alkyl group having 1 to 50 carbon atoms, a substituted or unsubstituted alkoxy group having 1 to 50 carbon atoms, a substituted or unsubstituted aralkyl group having 6 to 50 carbon atoms, a substituted or unsubstituted aryloxy group having 5 to 50 ring atoms, a substituted or unsubstituted arylthio group having 5 to 50 ring atoms, a substituted or unsubstituted alkoxycarbonyl group having 1 to 50 carbon atoms, a carboxyl group, a halogen atom, a cyano group, a nitro group, or a hydroxyl group;

a, b, and c each represent an integer of 0 to 4; and

n represents an integer of 1 to 3. In addition, when n represents 2 or more, anthracene nuclei in [ ] may be identical to or different from each other.)

an asymmetric monoanthracene derivative represented by the following formula (ii);

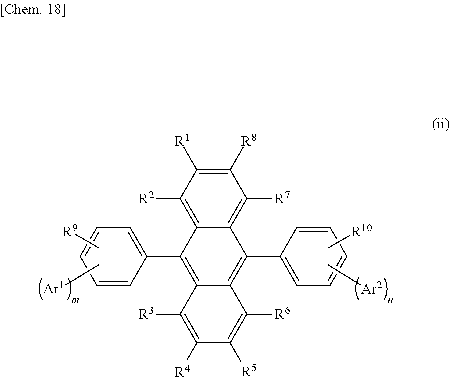

(In the formula: Ar¹ and Ar² each independently represent a substituted or unsubstituted aromatic ring group having 6 to 50 ring carbon atoms; m and n each represent an integer of 1 to 4, provided that Ar¹ and Ar² are not identical to each other when m=n=1 and positions at which Ar¹ and Ar² are bonded to a benzene ring are bilaterally symmetric, and m and n represent different integers when m or n represents an integer of 2 to 4; and

R¹ to R¹⁰ each independently represent a hydrogen atom, a substituted or unsubstituted aromatic ring group having 6 to 50 ring carbon atoms, a substituted or unsubstituted aromatic heterocyclic group having 5 to 50 ring atoms, a substituted or unsubstituted alkyl group having 1 to 50 carbon atoms, a substituted or unsubstituted cycloalkyl group, a substituted or unsubstituted alkoxy group having 1 to 50 carbon atoms, a substituted or unsubstituted aralkyl group having 6 to 50 carbon atoms, a substituted or unsubstituted aryloxy group having 5 to 50 ring atoms, a substituted or unsubstituted arylthio group having 5 to 50 ring atoms, a substituted or unsubstituted alkoxycarbonyl group having 1 to 50 carbon atoms, a substituted or unsubstituted silyl group, a carboxyl group, a halogen atom, a cyano group, a nitro group, or a hydroxyl group.)

an asymmetric pyrene derivative represented by the following formula (iii);

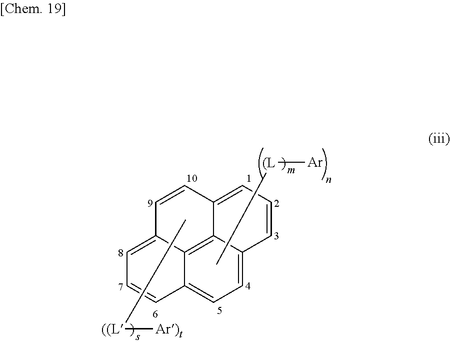

[In the formula: Ar and Ar′ each represent a substituted or unsubstituted aromatic group having 6 to 50 ring carbon atoms;

L and L′ each represent a substituted or unsubstituted phenylene group, a substituted or unsubstituted naphthalenylene group, a substituted or unsubstituted fluorenylene group, or a substituted or unsubstituted dibenzosilolylene group;

m represents an integer of 0 to 2, n represents an integer of 1 to 4; s represents an integer of 0 to 2, and t represents an integer of 0 to 4; and

in addition, L or Ar binds to any one of 1- to 5-positions of pyrene, and L′ or Ar′ binds to any one of 6- to 10-positions of pyrene,

provided that Ar, Ar′, L, and L′ satisfy the following item (1) or (2) when n+t represents an even number:

(1) Ar≠Ar′ and/or L≠L′ (where the symbol ‘≠’ means that groups connected with the symbol have different structures); and
(2) when Ar=Ar′ and L=L′,

(2-1) m≠s and/or n≠t, or

(2-2) when m=s and n=t,
(2-2-1) L and L′, or pyrene binds to different binding positions on Ar and Ar′, respectively, or (2-2-2) in the case where L and L′, or pyrene binds to the same binding positions on Ar and Ar′, the case where the substitution positions of L and L′, or of Ar and Ar′ in pyrene are 1- and 6-positions, or 2- and 7-positions is excluded.]

an asymmetric anthracene derivative represented by the following formula (Iv);

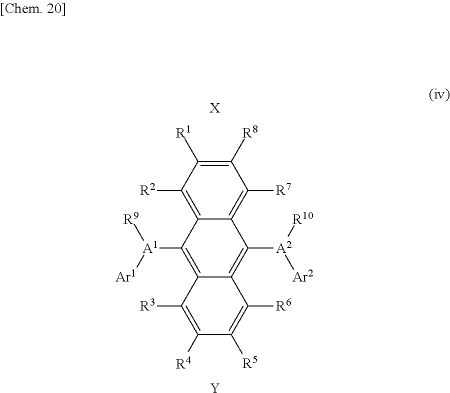

(In the formula: A¹ and A² each independently represent a substituted or unsubstituted fused aromatic ring group having 10 to 20 ring carbon atoms;

Ar¹ and Ar² each independently represent a hydrogen atom, or a substituted or unsubstituted aromatic ring group having 6 to 50 ring carbon atoms;

R¹ to R¹⁰ each independently represent a hydrogen atom, a substituted or unsubstituted aromatic ring group having 6 to 50 ring carbon atoms, a substituted or unsubstituted aromatic heterocyclic group having 5 to 50 ring atoms, a substituted or unsubstituted alkyl group having 1 to 50 carbon atoms, a substituted or unsubstituted cycloalkyl group, a substituted or unsubstituted alkoxy group having 1 to 50 carbon atoms, a substituted or unsubstituted aralkyl group having 6 to 50 carbon atoms, a substituted or unsubstituted aryloxy group having 5 to 50 ring atoms, a substituted or unsubstituted arylthio group having 5 to 50 ring atoms, a substituted or unsubstituted alkoxycarbonyl group having 1 to 50 carbon atoms, a substituted or unsubstituted silyl group, a carboxyl group, a halogen atom, a cyano group, a nitro group, or a hydroxyl group; and the number of each of Ar¹, Ar², R⁹, and R¹⁰ may be two or more, and adjacent groups may together form a saturated or unsaturated cyclic structure,

provided that the case where groups symmetric with respect to the X-Y axis shown on central anthracene in the general formula (1) bind to 9- and 10-positions of the anthracene is excluded.)

an anthracene derivative represented by the following formula (V);

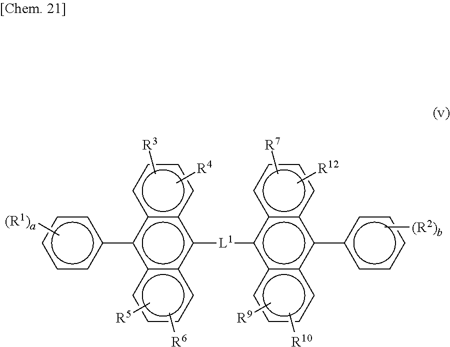

(In the formula: R¹ to R¹⁰ each independently represent a hydrogen atom, an alkyl group, a cycloalkyl group, an aryl group which may be substituted, an alkoxyl group, an aryloxy group, an alkylamino group, an alkenyl group, an arylamino group, or a heterocyclic group which may be substituted; a and b each represent an integer of 1 to 5, and, when a or b represents 2 or more, R¹'s or R²'s may be identical to or different from each other, respectively, or R¹'s or R²'s may be bonded to each other to form a ring; R³ and R⁴, R⁵ and R⁶, R⁷ and R⁸, or R⁹ and R¹⁰ may be bonded to each other to form a ring; and L¹ represents a single bond, —O—, —S—, —N(R)— (where R represents an alkyl group or an aryl group which may be substituted), an alkylene group, or an arylene group.)

an anthracene derivative represented by the following formula (Vi);

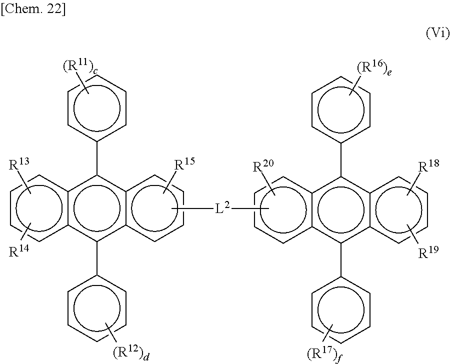

(In the formula: R¹¹ to R²⁰ each independently represent a hydrogen atom, an alkyl group, a cycloalkyl group, an aryl group, an alkoxyl group, an aryloxy group, an alkylamino group, an arylamino group, or a heterocyclic group which may be substituted; c, d, e, and f each represent an integer of 1 to 5, and, when any one of c, d, e, and f represents 2 or more, R¹¹'s, R¹²'s, R¹⁶'s, or R¹⁷'s may be identical to or different from each other, respectively, or R¹¹'s, R¹²'s, R¹⁶'s, or R¹⁷'s may be bonded to each other to form a ring; R¹³ and R^{N}, or R¹⁸ and R¹⁹ may be bonded to each other to form a ring; and L² represents a single bond, —O—, —S—, —N(R)— (where R represents an alkyl group or an aryl group which may be substituted), an alkylene group, or an arylene group.)

a spirofluorene derivative represented by the following formula (Vii);

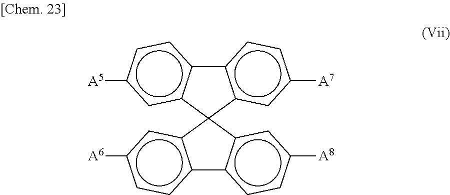

(In the formula, A⁵ to A⁸ each independently represent a substituted or unsubstituted biphenyl group, or a substituted or unsubstituted naphthyl group.)

a fused ring-containing compound represented by the following formula (Viii); and

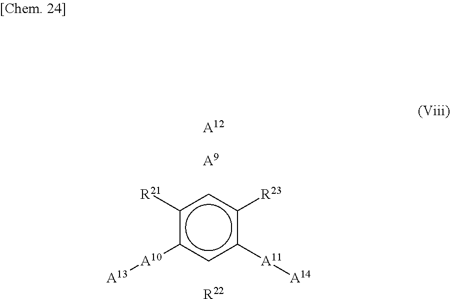

(In the formula, A⁹ to A¹⁴ each have the same meaning as that described above; R²¹ to R²³ each independently represent a hydrogen atom, an alkyl group having 1 to 6 carbon atoms, a cycloalkyl group having 3 to 6 carbon atoms, an alkoxyl group having 1 to 6 carbon atoms, an aryloxy group having 5 to 18 carbon atoms, an aralkyloxy group having 7 to 18 carbon atoms, an arylamino group having 5 to 16 carbon atoms, a nitro group, a cyano group, an ester group having 1 to 6 carbon atoms, or a halogen atom; and at least one of A⁹ to A¹⁴ represents a group having three or more fused aromatic rings.)

a fluorene compound represented by the following formula (ix).

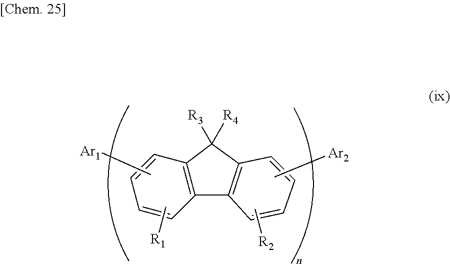

(In the formula: R₁ and R₂ each represent a hydrogen atom, a substituted or unsubstituted alkyl group, a substituted or unsubstituted aralkyl group, a substituted or unsubstituted aryl group, a substituted or unsubstituted heterocyclic group, a substituted amino group, a cyano group, or a halogen atom; R₁'s or R₂'s bonded to different fluorene groups may be identical to or different from each other, and R₁ and R₂ bonded to the same fluorene group may be identical to or different from each other; R₃ and R₄ each represent a hydrogen atom, a substituted or unsubstituted alkyl group, a substituted or unsubstituted aralkyl group, a substituted or unsubstituted aryl group, or a substituted or unsubstituted heterocyclic group; R₃'s or R₄'s bonded to different fluorene groups may be identical to or different from each other, and R₃ and R₄ bonded to the same fluorene group may be identical to or different from each other; Ar₁ and Ar₂ each represent a substituted or unsubstituted fused polycyclic aromatic group having three or more benzene rings in total, or a substituted or unsubstituted fused polycyclic heterocyclic group that has three or more rings each of which is a benzene ring or a heterocyclic ring in total and that is bonded to a fluorene group by carbon, and Ar₁ and Ar₂ may be identical to or different from each other; and n represents an integer of 1 to 10.)

Of the above-mentioned host materials (i) to (ix), an anthracene derivative is preferred, a monoanthracene derivative is more preferred, and an asymmetric anthracene is particularly preferred.

In addition, a phosphorescent compound can also be used as a dopant light emitting material. The nitrogen-containing heterocyclic derivative of the present invention or a derivative of the nitrogen-containing heterocyclic ring and/or a compound containing a carbazole ring as a host material is preferred as the phosphorescent compound. The dopant is a compound capable of emitting light from a triplet exciton, and is not particularly limited as long as light is emitted from a triplet exciton, a metal complex containing at least one metal selected from the group consisting of Ir, Ru, Pd, Pt, Os, and Re is preferred, and a porphyrin metal complex or an orthometalated metal complex is preferred.

A host compound formed of a compound containing a carbazole ring and suitable for phosphorescence is a compound having a function of causing a phosphorescent compound to emit light as a result of the occurrence of energy transfer from the excited state of the host to the phosphorescent compound. The host compound is not particularly limited as long as the host compound is a compound capable of transferring exciton energy to a phosphorescent compound, and can be appropriately selected in accordance with a purpose. The host compound may have, for example, an arbitrary heterocyclic ring in addition to a carbazole ring.

Specific examples of such host compound include a carbazole derivative, a triazole derivative, an oxazole derivative, an oxadiazole derivative, an imidazole derivative, a polyarylalkane derivative, a pyrazoline derivative, a pyrazolone derivative, a phenylenediamine derivative, an arylamine derivative, an amino substituted chalcone derivative, a styrylanthracene derivative, a fluorenone derivative, a hydrazone derivative, a stilbene derivative, a silazane derivative, an aromatic tertiary amine compound, a styrylamine compound, an aromatic dimethylidene-based compound, a porphyrin-based compound, an anthraquinodimethane derivative, an anthrone derivative, a diphenylquinone derivative, a thiopyranedioxide derivative, a carbodiimide derivative, a fluorenilidenemethane derivative, a distyrylpyrazine derivative, a heterocyclic tetracarboxylic anhydride such as naphthaleneperylene, a phthalocyanine derivative, various metal complex polysilane-based compounds typified by a metal complex of an 8-quinolinol derivative or a metal complex having metal phthalocyanine, benzooxazole, or benzothiazole as a ligand, a poly(N-vinylcarbazole) derivative, an aniline-based copolymer, a conductive high molecular weight oligomer such as a thiophene oligomer or polythiophene, and polymer compounds such as a polythiophene derivative, a polyphenylene derivative, a polyphenylene vinylene derivative, and a polyfluorene derivative. One of the host materials may be used alone, or two or more kinds of them may be used in combination.

Specific examples thereof include the compounds as described below.

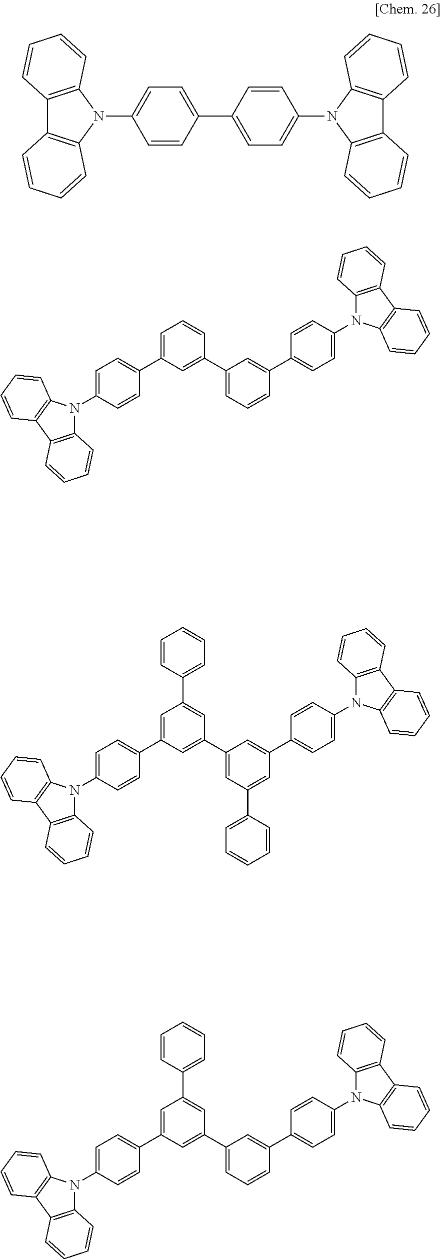

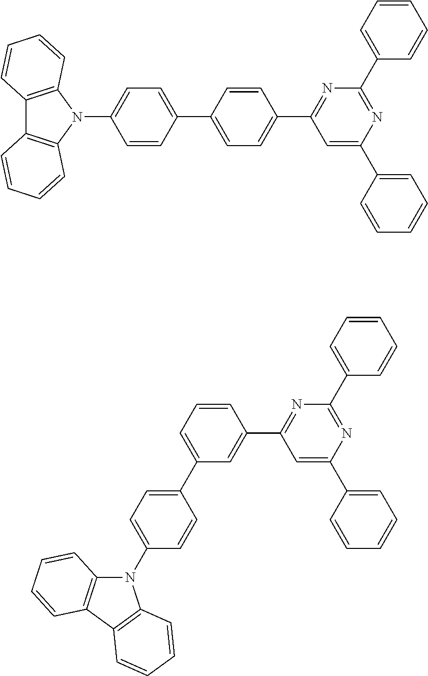

A phosphorescent dopant is a compound capable of emitting light from a triplet exciton. The dopant, which is not particularly limited as long as light is emitted from a triplet exciton, is preferably a metal complex containing at least one metal selected from the group consisting of Ir, Ru, Pd, Pt, Os, and Re, and is preferably a porphyrin metal complex or an orthometalated metal complex. A porphyrin platinum complex is preferred as the porphyrin metal complex. One kind of a phosphorescent compound may be used alone, or two or more kinds of phosphorescent compounds may be used in combination.

Any one of various ligands can be used for forming an orthometalated metal complex. A preferred ligand is, for example, a 2-phenylpyridine derivative, a 7,8-benzoquinoline derivative, a 2-(2-thienyl)pyridine derivative, a 2-(1-naphthyl)pyridine derivative, or a 2-phenylquinoline derivative. Each of those derivatives may have a substituent as required. A fluoride of any one of those derivatives, or one obtained by introducing a trifluoromethyl group into any one of those derivatives is a particularly preferred blue-based dopant. The metal complex may further include a ligand other than the above-mentioned ligands, such as acetylacetonato or picric acid as an auxiliary ligand.

The content of the phosphorescent dopant in the light emitting layer is not particularly limited, and can be appropriately selected in accordance with a purpose. The content is, for example, 0.1 to 70 mass %, preferably 1 to 30 mass %. When the content of the phosphorescent compound is less than 0.1 mass %, the intensity of emitted light is weak, and an effect of the incorporation of the compound is not sufficiently exerted. When the content exceeds 70 mass %, a phenomenon referred to as concentration quenching becomes remarkable, and device performance reduces.

In addition, the light emitting layer may contain a hole transporting material, an electron transporting material, or a polymer binder as required.

Further, the thickness of the light emitting layer is preferably 5 to 50 nm, more preferably 7 to 50 nm, most preferably 10 to 50 nm. When the thickness is less than 5 nm, it becomes difficult to form the light emitting layer, and hence the adjustment of chromaticity may be difficult. When the thickness exceeds 50 nm, the driving voltage may increase.

(Hole Injecting/Transporting Layer (Hole Transporting Zone))

The hole injecting/transporting layer is a layer which helps injection of holes into the light emitting layer and transports the holes to the light emitting region. The layer exhibits a great mobility of holes and, in general, has an ionization energy as small as 5.5 eV or smaller. For such hole injecting/transporting layer, a material which transports holes to the light emitting layer under an electric field of a smaller strength is preferred. Further, the material preferably exhibits, for example, a mobility of holes of at least 10⁻⁴ cm²/V·sec under application of an electric field of 10⁴ to 10⁶ V/cm.

A hole injecting material or hole transporting material for an organic electroluminescence device containing the nitrogen-containing heterocyclic derivative of the present invention can be used as a material for forming the hole injecting/transporting layer of the organic EL device of the present invention. In addition, an arbitrary material selected from materials conventionally used as hole charge transporting materials in photoconductive materials and known materials used in the hole injecting/transporting layers of organic EL devices can be used in combination with, or instead of, the hole injecting material or hole transporting material for an organic electroluminescence device of the present invention.

Specific examples of the known materials include: a triazole derivative (see, for example, U.S. Pat. No. 3,112,197 A); an oxadiazole derivative (see, for example, U.S. Pat. No. 3,189,447 A); an imidazole derivative (see, for example, JP 37-16096 B); a polyarylalkane derivative (see, for example, U.S. Pat. No. 3,615,402 A, U.S. Pat. No. 3,820,989 A, U.S. Pat. No. 3,542,544 A, JP 45-555 B, JP 51-10983 B, JP 51-93224 A, JP 55-17105 A, JP 56-4148 A, JP 55-108667 A, JP 55-156953 A, and JP 56-36656 A); a pyrazoline derivative and a pyrazolone derivative (see, for example, U.S. Pat. No. 3,180,729 A, U.S. Pat. No. 4,278,746 A, JP 55-88064 A, JP 55-88065 A, JP 49-105537 A, JP 55-51086 A, JP 56-80051 A, JP 56-88141 A, JP 57-45545 A, JP 54-112637 A, and JP 55-74546 A); a phenylenediamine derivative (see, for example, U.S. Pat. No. 3,615,404 A, JP 51-10.105 B, JP 46-3712 B, JP 47-25336 B, JP 54-53435 A, JP 54-110536 A, and JP 54-119925 A); an arylamine derivative (see, for example, U.S. Pat. No. 3,567,450 A, U.S. Pat. No. 3,180,703 A, U.S. Pat. No. 3,240,597 A, U.S. Pat. No. 3,658,520 A, U.S. Pat. No. 4,232,103 A, U.S. Pat. No. 4,175,961 A, U.S. Pat. No. 4,012,376 A, JP 49-35702 B, JP 39-27577 B, JP 55-144250 A, JP 56-119132 A, JP 56-22437 A, and DE 1110518 C); an amino-substituted chalcone derivative (see, for example, US 352650.1 A); an oxazole derivative (those disclosed in, for example, U.S. Pat. No. 3,257,203 A); a styrylanthracene derivative (see, for example, JP 56-46234 A); a fluorenone derivative (see, for example, JP 54-110837 A); a hydrazone derivative (see, for example, U.S. Pat. No. 3,717,462 A, JP 54-59143 A, JP 55-52063 A, JP 55-52064 A, JP 55-46760 A, JP 55-85495 A, JP 57-11350 A, JP 57-148749 A, and JP 02-311591 A); a stilbene derivative (see, for example, JP 61-210363 A, JP 61-228451 A, JP 61-14642 A, JP 61-72255 A, JP 62-47646 A, JP 62-36674 A, JP 62-10652 A, JP 62-30255 A, JP 60-93445 A, JP 60-94462 A, JP 60-174749 A, and JP 60-175052 A); a silazane derivative (U.S. Pat. No. 4,950,950 A); a polysilane-based copolymer (JP 02-204996 A); an aniline-based copolymer (JP 02-282263 A); and a conductive high molecular weight oligomer as described in JP 01-211399 A (in particular, a thiophene oligomer).

In addition to the above-mentioned materials which can be used as the material for the hole injecting/transporting layer, a porphyrin compound (those disclosed in, for example, JP 63-295695 A); an aromatic tertiary amine compound and a styrylamine compound (see, for example, U.S. Pat. No. 4,127,412 A, JP 53-27033 A, JP 54-58445 A, JP 54-149634 A, JP 54-64299 A, JP 55-79450 A, JP 55-144250 A, JP 56-119132 A, JP 61-295558 A, JP 61-98353 A, and JP 63-295695 A) are preferred, and an aromatic tertiary amine compound is particularly preferred.

Further, examples of the aromatic tertiary amine compound include compounds each having two fused aromatic rings in the molecule, such as 4,4′-bis(N-(1-naphthyl)-N-phenylamino)-biphenyl (hereinafter, abbreviated as “NPD”) as described in U.S. Pat. No. 5,061,569 A, and a compound in which three triphenylamine units are bonded together in a star-burst shape, such as 4,4′,4″-tris(N-(3-methylphenyl)-N-phenylamino)triphenylamine (hereinafter, abbreviated as “MTDATA”) as described in JP 04-308688 A.

Further, in addition to the aromatic dimethylidene-based compounds described above as the material for the light emitting layer, inorganic compounds such as p-type Si and p-type SiC can also be used as the material for the hole injecting/transporting layer.

In addition, the hole injecting/transporting layer preferably further contains a hole injecting substance selected from the group consisting of a phthalocyanine copper complex compound, an oligothiophene, an arylamine-based compound, and a polycyclic aromatic compound.

The hole injecting/transporting layer can be formed by forming the hole injecting/transporting material into a thin layer in accordance with a known process such as a vacuum vapor deposition process, a spin coating process, a casting process, or an LB process. The thickness of the hole injecting/transporting layer is not particularly limited. In general, the thickness is 5 nm to 5 μm. The hole injecting/transporting layer may be formed of a single layer formed of one kind or two or more kinds of the materials described above or may be a laminate formed of hole injecting/transporting layers containing materials different from the materials of the hole injecting/transporting layer described above as long as the hole injecting/transporting material is incorporated in the hole transporting zone.

Further, an organic semiconductor layer may be disposed as a layer for helping the injection of holes or electrons into the light emitting layer. As the organic semiconductor layer, a layer having a conductivity of 10⁻¹⁰ S/cm or more is preferred. As the material for the organic semiconductor layer, oligomers each containing a thiophene, and conductive oligomers such as oligomers each containing an arylamine and conductive dendrimers such as dendrimers each containing an arylamine, which are disclosed in JP 08-193191 A, can be used.

(Electron Injecting/Transporting Layer (Electron Transporting Zone))

The electron injecting/transporting layer is a layer which helps injection of electrons into the light emitting layer, transports the electrons to the light emitting region, and exhibits a great mobility of electrons. In addition, the adhesion improving layer is an electron injecting layer including a material exhibiting particularly improved adhesion with the cathode. In the organic EL device of the present invention, an electron injecting material or electron transporting material for an organic electroluminescence device containing the nitrogen-containing heterocyclic derivative of the present invention is preferably used in the electron injecting layer or transporting layer, or the adhesion improving layer.

When the nitrogen-containing heterocyclic derivative of the present invention is used in the electron transporting zone, the electron injecting or transporting layer may be formed of the nitrogen-containing heterocyclic derivative of the present invention alone, or the derivative may be used as a mixture or laminate with any other material.

The material that is mixed or laminated with the nitrogen-containing heterocyclic derivative of the present invention to form the electron injecting/transporting layer is not particularly limited as long as the material has the preferred nature, and an arbitrary material selected from materials conventionally used as electron charge transporting materials in photoconductive materials and known materials used in the electron injecting/transporting layers of organic EL devices can be used.

A preferred embodiment of the organic EL device of the present invention includes a device including a reduction-causing dopant in the region of electron transport or in the interfacial region of the cathode and the organic layer. In the present invention, an organic EL device containing a reduction-causing dopant in the nitrogen-containing heterocyclic derivative of the present invention is preferred. Here, the reduction-causing dopant is defined as a substance which can reduce a compound having electron transporting property. Therefore, various compounds can be used as the reduction-causing dopant as long as the compounds have a certain level of reductive property. For example, at least one substance selected from the group consisting of alkali metals, alkaline earth metals, rare earth metals, oxides of alkali metals, halides of alkali metals, oxides of alkaline earth metals, halides of alkaline earth metals, oxides of rare earth metals, halides of rare earth metals, organic complexes of alkali metals, organic complexes of alkaline earth metals, and organic complexes of rare earth metals can be preferably used.

More specifically, preferred examples of the reduction-causing dopant include substances each having a work function of particularly preferably 2.9 eV or less, and specific examples of which include at least one alkali metal selected from the group consisting of Na (the work function: 2.36 eV), K (the work function: 2.28 eV), Rb (the work function: 2.16 eV), and Cs (the work function: 1.95 eV) and at least one alkaline earth metal selected from the group consisting of Ca (the work function: 2.9 eV), SR (the work function: 2.0 to 2.5 eV), and Ba (the work function: 2.52 eV). Of those, at least one alkali metal selected from the group consisting of K, Rb, and Cs is more preferred, Rb and Cs are still more preferred, and Cs is most preferred as the reduction-causing dopant. Those alkali metals each have particularly great reducing ability, and the luminance of the emitted light and the life time of the organic EL device can be increased by addition of a relatively small amount of the alkali metal into the electron injecting zone. In addition, as the reduction-causing dopant having a work function of 2.9 eV or less, a combination of two or more kinds of those alkali metals is also preferred. Combinations including Cs such as the combinations of Cs and Na, Cs and K, Cs and Rb, and Cs, Na, and K are particularly preferred. The reducing ability can be efficiently exhibited by the combination including Cs. The luminance of emitted light and the life time of the organic EL device can be increased by adding the combination including Cs into the electron injecting zone.

The present invention may further include an electron injecting layer which is formed of an insulating material or a semiconductor and disposed between the cathode and the organic layer. In this case, the electron injecting property can be improved by preventing a leak of electric current effectively. As the insulating material, at least one metal compound selected from the group consisting of alkali metal chalcogenides, alkaline earth metal chalcogenides, halides of alkali metals, and halides of alkaline earth metals is preferably used. It is preferred that the electron injecting layer be formed of the above-mentioned substance such as the alkali metal chalcogenide because the electron injecting property can be further improved. Specifically, preferred examples of the alkali metal chalcogenide include Li₂O, K₂O, Na₂S, Na₂Se, and Na₂O. Preferred examples of the alkaline earth metal chalcogenide include CaO, BaO, SrO, BeO, BaS, and CaSe. In addition, preferred examples of the alkali metal halide include LiF, NaF, KF, LiCl, KCl, and NaCl. In addition, preferred examples of the alkaline earth metal halide include fluorides such as CaF₂, BaF₂, SrF₂, MgF₂, and BeF₂ and halides other than the fluorides.

In addition, examples of the semiconductor forming the electron transporting layer include oxides, nitrides, and oxide nitrides each containing at least one element selected from Ba, Ca, Sr, Yb, Al, Ga, In, Li, Na, Cd, Mg, Si, Ta, Sb, and Zn used alone or in combination of two or more kinds. In addition, it is preferred that the inorganic compound forming the electron transporting layer form a crystallite or amorphous insulating thin film. When the electron transporting layer is formed of the insulating thin film described above, a more uniform thin film can be formed, and hence defects of pixels such as dark spots can be decreased. It should be noted that examples of the inorganic compound include the alkali metal chalcogenides, alkaline earth metal chalcogenides, halides of alkali metals, and halides of alkaline earth metals which are described above.

(Cathode)

As the cathode, one using, as an electrode material, a material such as a metal, an alloy, an electroconductive compound, or a mixture of those materials which has a small work function (4 eV or less) is used because the cathode is used for injecting electrons to the electron injecting/transporting layer or the light emitting layer. Specific examples of the electrode material include sodium, sodium-potassium alloys, magnesium, lithium, magnesium-silver alloys, aluminum/aluminum oxide, aluminum-lithium alloys, indium, and rare earth metals.

The cathode can be prepared by forming a thin film of the electrode material described above in accordance with a process such as a vapor deposition process or a sputtering process.

Here, in the case of a top surface emission-type or top emission-type organic EL device, it is preferred that the transmittance of the cathode be more than 10% with respect to the emitted light.

It is also preferred that the sheet resistivity of the cathode be several hundred Ω/□ or less. The thickness of the cathode is, in general, 10 nm to 1 μm, preferably 50 to 200 nm.

(Insulating Layer)

Defects in pixels tend to be formed in organic EL device due to leak and short circuit because an electric field is applied to ultra-thin films. In order to prevent the formation of the defects, a layer of a thin film having insulating property may be inserted between the pair of electrodes.

Examples of the material to be used for the insulating layer include aluminum oxide, lithium fluoride, lithium oxide, cesium fluoride, cesium oxide, magnesium oxide, magnesium fluoride, calcium oxide, calcium fluoride, aluminum nitride, titanium oxide, silicon oxide, germanium oxide, silicon nitride, boron nitride, molybdenum oxide, ruthenium oxide, and vanadium oxide. Mixtures and laminates of the above-mentioned materials may also be used.

(Method of Producing Organic EL Device)

The organic EL device can be fabricated by forming the anode and the light emitting layer, and, as required, the hole injecting/transporting layer and the electron injecting/transporting layer and further forming the cathode in accordance with the illustrated process using the illustrated materials. The organic EL device may also be fabricated by forming the above-mentioned layers in the order reverse to the order described above, i.e., the cathode being formed in the first step and the anode in the last step.

Hereinafter, an example of the production of an organic EL device having a configuration in which an anode, a hole injecting layer, a light emitting layer, an electron injecting layer, and a cathode are disposed successively on a light-transmissive substrate is described.

First, on a suitable light-transmissive substrate, a thin film made of a material for the anode is formed in accordance with the vapor deposition process or the sputtering process so that the thickness of the formed thin film is 1 μm or less, preferably in the range of 10 to 200 nm. The formed thin film is used as the anode. Then, a hole injecting layer is formed on the anode. The hole injecting layer can be formed in accordance with the vacuum vapor deposition process, the spin coating process, the casting process, or the LB process, as described above. The vacuum vapor deposition process is preferred because a uniform film can be easily obtained and the possibility of formation of pin holes is small. When the hole injecting layer is formed in accordance with the vacuum vapor deposition process, in general, it is preferred that the conditions be suitably selected in the following ranges: the temperature of the source of the deposition: 50 to 450° C.; the degree of vacuum: 10⁻⁷ to 10⁻³ Torr; the rate of deposition: 0.01 to 50 nm/second; the temperature of the substrate: −50 to 300° C.; and the thickness of the film: 5 nm to 5 μm although the conditions of the vacuum vapor deposition vary depending on the compound to be used (i.e., material for the hole injecting layer) and the crystal structure and recombination structure of the target hole injecting layer.

Then, the light emitting layer is formed on the hole injecting layer. The light emitting layer can also be formed by forming a desired organic light emitting material into a thin film in accordance with a process such as the vacuum vapor deposition process, the sputtering process, the spin coating process, or the casting process. The vacuum vapor deposition process is preferred because a uniform film can be easily obtained and the possibility of formation of pin holes is small. When the light emitting layer is formed in accordance with the vacuum vapor deposition process, in general, the conditions of the vacuum vapor deposition process can be selected in the same ranges as the condition ranges described for the hole injecting layer, although the conditions vary depending on the compound to be used.

Next, an electron injecting layer is formed on the light emitting layer. Similarly to the hole injecting layer and the light emitting layer, it is preferred that the electron injecting layer be formed in accordance with the vacuum vapor deposition process because a uniform film must be obtained. The conditions of the vacuum vapor deposition can be selected in the same ranges as the condition ranges described for the hole injecting layer and the light emitting layer.

When the vapor deposition process is used, the nitrogen-containing heterocyclic derivative of the present invention can be deposited from the vapor in combination with other materials, although the situation may be different depending on which layer in the light emitting zone or in the electron injecting zone or the electron transporting zone contains the derivative. In addition, when the spin coating process is used, the derivative can be incorporated into the formed layer by using a mixture of the derivative with other materials.

A cathode is laminated in the last step, and an organic EL device can be obtained.

The cathode is formed of a metal and can be formed in accordance with the vacuum vapor deposition process or the sputtering process. It is preferred that the vacuum vapor deposition process be used in order to prevent formation of damages on the lower organic layers during the formation of the film.

In the above-mentioned production of the organic EL device, it is preferred that the above-mentioned layers from the anode to the cathode be formed successively while the production system is kept in a vacuum after being evacuated once.

A method of forming each layer in the organic EL device of the present invention is not particularly limited. A conventionally known process such as a vacuum vapor deposition process or a spin coating process can be used. The organic thin film layer which is used in the organic EL device of the present invention and contains the nitrogen-containing heterocyclic derivative represented by the formula (1) described above can be formed in accordance with a known process such as a vacuum vapor deposition process or a molecular beam epitaxy process (MBE process) or, using a solution prepared by dissolving the derivative into a solvent, in accordance with a coating process such as a dipping process, a spin coating process, a casting process, a bar coating process, or a roll coating process.

The thickness of each organic layer in the organic EL device of the present invention is not particularly limited. In general, an excessively thin layer tends to have defects such as pin holes, whereas an excessively thick layer requires a high applied voltage to decrease the efficiency. Therefore, a thickness in the range of several nanometers to 1 μm is preferred.

It should be noted that the organic EL device emits light when a DC voltage of 5 to 40 V is applied in the condition that the polarity of the anode is positive (+) and the polarity of the cathode is negative (−). In addition, when the polarity is reversed, no electric current is observed and no light is emitted at all. Further, when an AC voltage is applied to the organic EL device, uniform light emission is observed only in the condition that the polarity of the anode is positive and the polarity of the cathode is negative. When an alternating voltage is applied to the organic EL device, any type of wave shape can be used.

(Application of Organic EL Device)

The organic EL device of the present invention can be applied to a product requested to show high brightness and high luminous efficiency even at a low voltage. Examples of the application include a display apparatus, a lighting apparatus, a printer light source, and a backlight for a liquid crystal display apparatus. The display apparatus is, for example, a flat panel display that has achieved energy savings or high visibility. In addition, with regard to the printer light source, the device can be used as a light source for a laser beam printer. In addition, the use of the device of the present invention can significantly reduce an apparatus volume. With regard to the lighting apparatus and the backlight, an energy-saving effect can be expected from the use of the organic EL device of the present invention.

In addition, the nitrogen-containing heterocyclic derivative of the present invention can find applications in materials for organic solar cells and organic semiconductors.

### EXAMPLES

Next, the present invention is described in more detail by way of examples. However, the present invention is by no means limited by these examples.

### Synthesis Example 1

### Synthesis of Compound 1

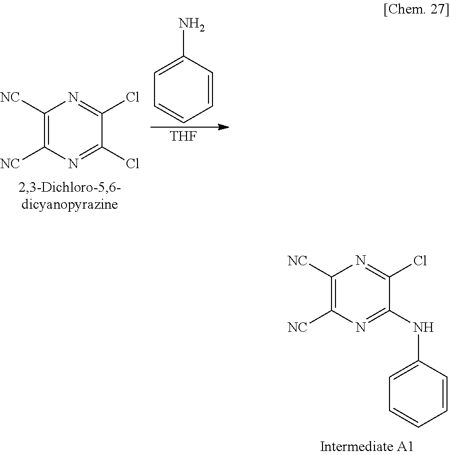

Compound 1 was synthesized with reference to the method described in J. Heterocyclic. Chem., vol. 34, p. 653, 1997. That is, 5.0 g (25 mmol) of 2,3-dichloro-5,6-dicyanopyrazine were dissolved in 100 mL of tetrahydrofuran in a 300-mL flask. Then, a solution of 5.9 g (63 mmol) of aniline in 50 mL of tetrahydrofuran was slowly dropped to the resultant solution while the latter solution was cooled to −20 to −40° C. After the completion of the dropping, the mixture was stirred for about an additional thirty minutes. After the completion of the reaction, the reaction mixture was poured into ice water, and then the precipitated crystal was taken by filtration. The resultant crystal was sufficiently washed with water, and was then further washed with a small amount of ethanol. After that, the crystal was dried under reduced pressure. Thus, 6.4 g of Intermediate A1 were obtained (99%). Intermediate A1 can be purified by recrystallization from ethanol as required.

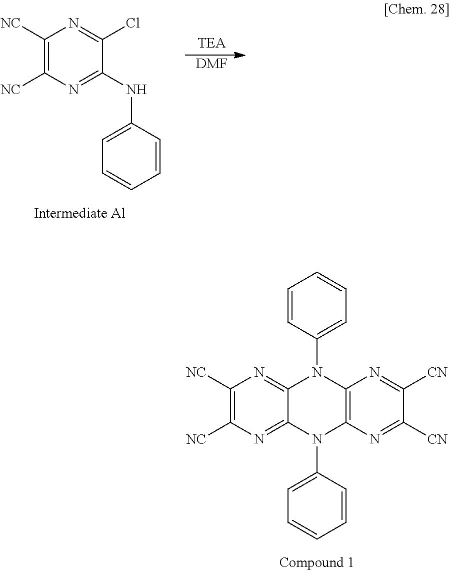

6.4 g (25 mmol) of Intermediate A1 thus obtained were dissolved in 100 mL of N,N-dimethylformamide (DMF). 5.6 g (55 mmol) of triethylamine (TEA) were added to the solution, and then the mixture was heated at 140° C. for 8 hours. After the completion of the reaction, the reaction mixture was poured into a 5% aqueous solution of hydrochloric acid, and then the precipitated crystal was taken by filtration. The resultant crystal was sufficiently washed with water, and was then dried under reduced pressure. Thus, a crude reaction product was obtained. The resultant crude reaction product was crystallized and washed with ethyl acetate. Thus, 3.9 g of Compound 1 were obtained as a yellow solid (in 36% yield). The solid was identified as Compound 1 by field desorption mass spectrometry (FD-MS).

### Synthesis Example 2

### Synthesis of Compound 2

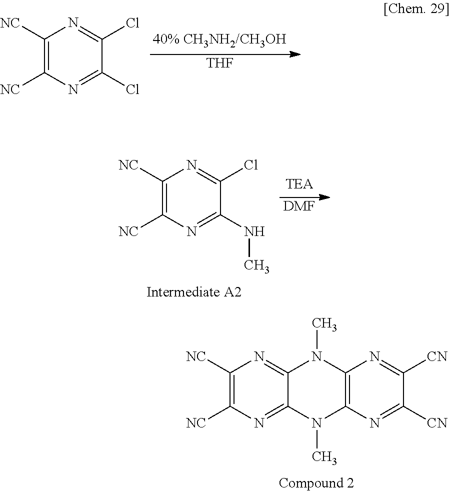

Compound 2 was obtained in 42% yield by performing the same operations as those of Synthesis Example 1 except that a 40% solution of methylamine in methanol was used instead of aniline. The resultant was identified as Compound 2 by FD-MS.

### Synthesis Example 3

### Synthesis of Compound 3

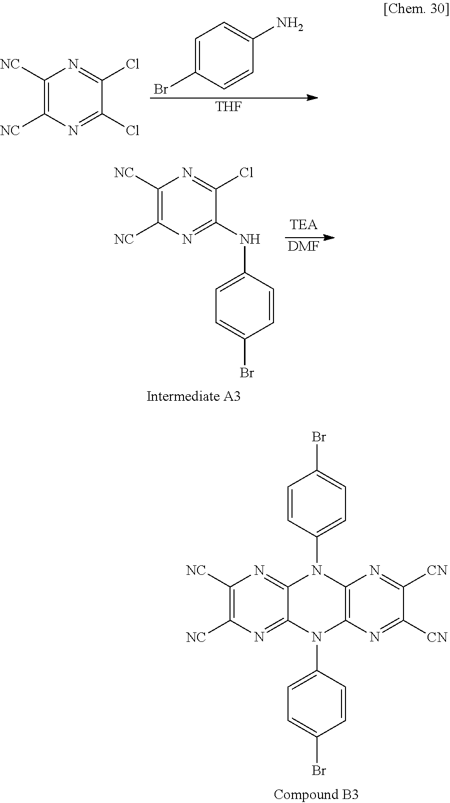

Compound B3 was obtained in 45% yield by performing the same operations as those of Synthesis Example 1 except that 4-bromoaniline was used instead of aniline.

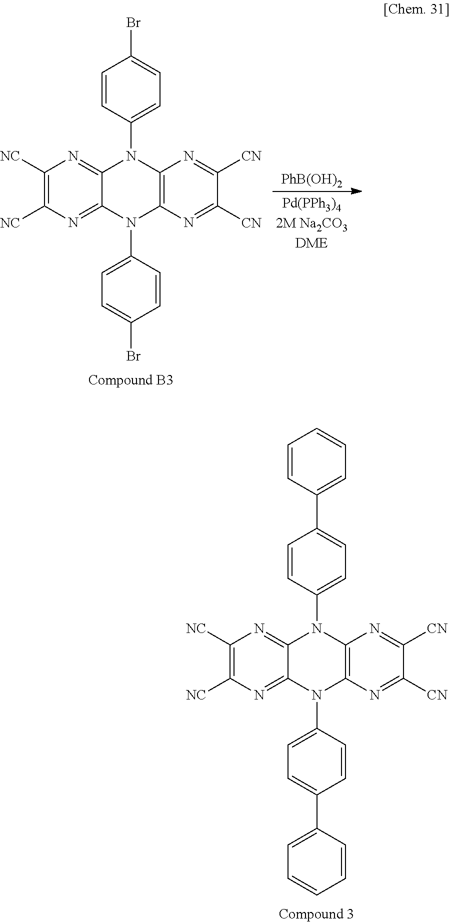

In a stream of argon, 3.0 g (5.0 mmol) of Compound B3, 1.4 g (11 mmol) of phenylboronic acid, 0.24 g (0.21 mmol) of tetrakistriphenylphosphinepalladium(0), 40 mL of 1,2-dimethoxyethane, and 18 mL of a 2-M aqueous solution of sodium carbonate were added into a 300-mL three-necked flask, and then the mixture was refluxed under heat for 8 hours. After the completion of the reaction, water was added. The precipitated solid was washed with water, and was then further washed with a small amount of ethanol. The resultant solid was purified by recrystallization from ethyl acetate. Thus, 1.5 g of a yellow powder were obtained in 50% yield. The powder was identified as Compound 3 by FD-MS.

### Synthesis Example 4

### Synthesis of Compound 4

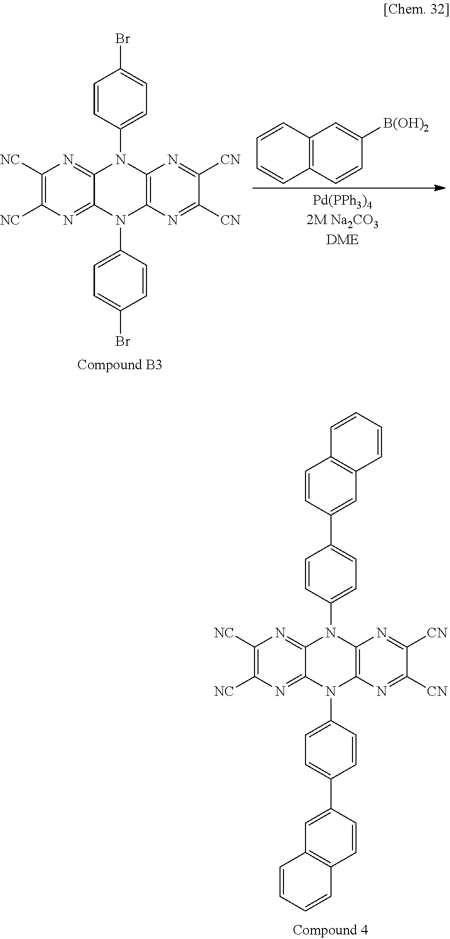

Compound 4 was obtained in 55% yield by performing the same operations as those of Synthesis Example 3 except that 2-naphthylboronic acid was used instead of phenylboronic acid. The resultant was identified as Compound 4 by FD-MS.

### Example 1

### Production of Organic EL Device Using Nitrogen-Containing Heterocyclic Derivative of the Present Invention in its Hole Injecting Layer

A glass substrate measuring 25 mm wide by 75 mm long by 1.1 mm thick and provided with an ITO transparent electrode (anode) (manufactured by GEOMATEC Co., Ltd.) was subjected to ultrasonic cleaning in isopropyl alcohol for 5 minutes. After that, the resultant was subjected to UV ozone cleaning for 30 minutes. The glass substrate provided with a transparent electrode line after the cleaning was mounted on a substrate holder of a vacuum deposition device, and, first, Compound 1 of the present invention as a hole injecting material was formed into a film having a thickness of 10 nm to serve as a hole injecting layer on the surface on the side where the transparent electrode line was formed so as to cover the transparent electrode. Subsequently, a N,N′-bis(1-naphthyl)-N,N′-diphenylbenzidine (NPD) film having a thickness of 70 nm was formed on the film. The NPD film functions as a hole transporting layer.

Further, Host compound H1 and Styrylamine derivative D1 represented by the following formulae were formed into a film having a thickness of 40 nm at a thickness ratio of 37:3 on the NPD film, to thereby obtain a bluish light emitting layer.

Tris(8-quinolinolato)aluminum (Alq) was formed by vapor deposition into a film having a thickness of 20 nm to serve as an electron transporting layer on the film. After that, LiF was formed into a film having a thickness of 1 nm. Metal Al was deposited from the vapor onto the LiF film to form a metal cathode having a thickness of 150 nm. Thus an organic EL device was formed.

The voltage, luminous brightness, and current efficiency of the resultant organic EL device at a current density of 10.0 mA/cm² were measured, and its luminescent color was observed. Table 1 shows those results.

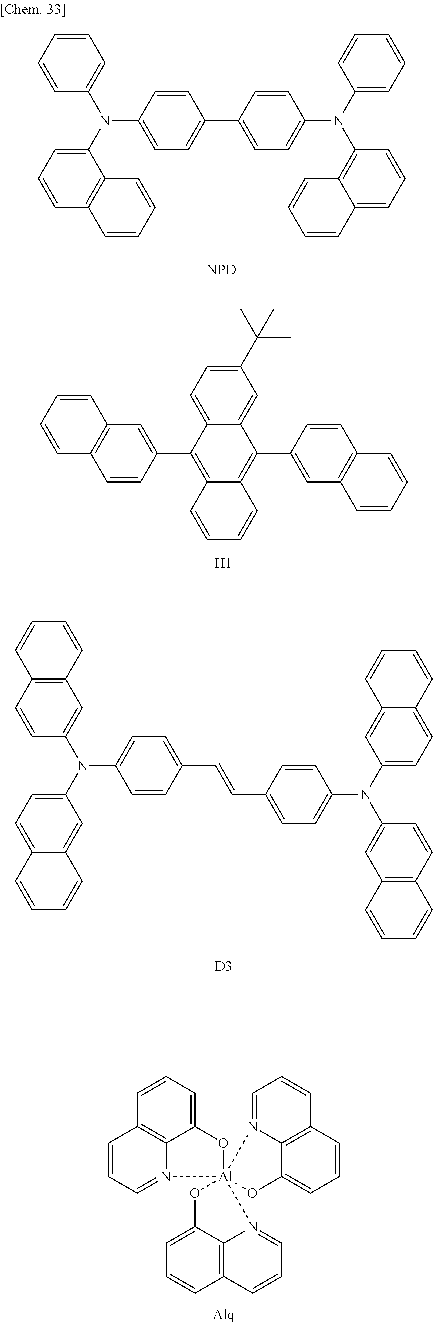

### Example 2

An organic EL device was fabricated in the same manner as in Example 1 except that Compound 2 was used instead of Compound 1. The voltage, luminous brightness, and current efficiency of the resultant organic EL device at a current density of 10.0 mA/cm² were measured, and its luminescent color was observed. Table 1 shows those results.

### Comparative Example 1

An organic EL device was fabricated in the same manner as in Example 1 except that Compound A below, which is described in JP 3614405 B2, was used instead of Compound 1. The voltage, luminous brightness, and current efficiency of the resultant organic EL device at a current density of 10.0 mA/cm² were measured, and its luminescent color was observed. Table 1 shows those results.

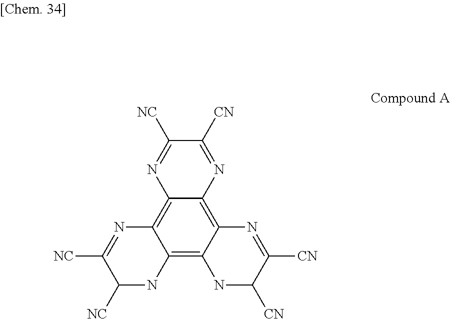

### Comparative Example 2

An organic EL device was fabricated in the same manner as in Example 1 except that N,N′-bis(N,N′-diphenyl-4-aminophenyl)-N,N-diphenyl-4,4′-diamino-1,1′-biphenyl (hereinafter, referred to as “TPD232”) was used instead of Compound 1. The voltage, luminous brightness, and current efficiency of the resultant organic EL device at a current density of 10.0 mA/cm² were measured, and its luminescent color was observed. Table 1 shows those results.

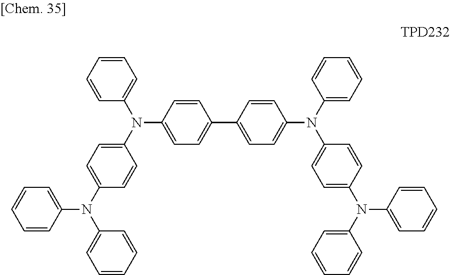

TABLE 1

| | Hole | Current | | Current | Lumi- |
|---|---|---|---|---|---|
| | injecting | density | Voltage | efficiency | nescent |
| | material | (mA/cm²) | (V) | (cd/A) | color |
| Example 1 | Compound 1 | 10.0 | 5.1 | 5.74 | Blue |
| Example 2 | Compound 2 | 10.0 | 4.8 | 5.10 | Blue |
| Comparative | Compound A | 10.0 | 5.3 | 5.28 | Blue |
| Example 1 | | | | | |
| Comparative | TPD232 | 10.0 | 6.2 | 4.80 | Blue |
| Example 2 | | | | | |

As can be seen from the results of Table 1, the use of the nitrogen-containing heterocyclic derivative of the present invention in a hole injecting layer enables the production of an organic EL device which can be driven at an extremely low voltage and shows high current efficiency.

### Example 3

### Production of Organic EL Device Using Nitrogen-Containing Heterocyclic Derivative of the Present Invention in its Electron Transporting Layer

A glass substrate measuring 25 mm wide by 75 mm long by 1.1 mm thick and provided with an ITO transparent electrode (anode) (manufactured by GEOMATEC Co., Ltd.) was subjected to ultrasonic cleaning in isopropyl alcohol for 5 minutes. After that, the resultant was subjected to UV ozone cleaning for 30 minutes. The glass substrate provided with a transparent electrode line after the cleaning was mounted on a substrate holder of a vacuum deposition device, and, first, a TPD232 film having a thickness of 60 nm was formed on the surface on the side where the transparent electrode line was formed so as to cover the transparent electrode. The TPD232 film functions as a hole injecting layer. Subsequent to the formation of the TPD232 film, an NPD film having a thickness of 20 nm was formed on the TPD232 film. The NPD film functions as a hole transporting layer.

Further, Host compound H1 and Styrylamine derivative D1 were formed into a film having a thickness of 40 nm at a thickness ratio of 37:3 on the NPD film, to thereby obtain a bluish light emitting layer.

Compound 1 of the present invention was formed by vapor deposition into a film having a thickness of 20 nm to serve as an electron transporting layer on the film. After that, LiF was formed into a film having a thickness of 1 nm. Metal Al was deposited from the vapor onto the LiF film to form a metal cathode having a thickness of 150 nm. Thus an organic EL device was formed.

The voltage, luminous brightness, and current efficiency of the resultant organic EL device at a current density of 10.0 mA/cm² were measured, and its luminescent color was observed. Table 2 shows those results.

### Example 4

An organic EL device was fabricated in the same manner as in Example 3 except that Compound 2 was used instead of Compound 1. The voltage, luminous brightness, and current efficiency of the resultant organic EL device at a current density of 10.0 mA/cm² were measured, and its luminescent color was observed. Table 2 shows those results.

### Comparative Example 3

An organic EL device was fabricated in the same manner as in Example 3 except that Alq was used instead of Compound 1. The voltage, luminous brightness, and current efficiency of the resultant organic EL device at a current density of 10.0 mA/cm² were measured, and its luminescent color was observed. Table 2 shows those results.

TABLE 2

| | Electron | Current | | Current | Lumi- |
|---|---|---|---|---|---|
| | transporting | density | Voltage | efficiency | nescent |
| | material | (mA/cm²) | (V) | (cd/A) | color |
| Example 3 | Compound 1 | 10.0 | 4.7 | 5.92 | Blue |
| Example 4 | Compound 2 | 10.0 | 5.0 | 5.05 | Blue |
| Comparative | Alq | 10.0 | 6.2 | 4.80 | Blue |
| Example 3 | | | | | |

As can be seen from the results of Table 2, the use of the nitrogen-containing heterocyclic derivative of the present invention in an electron transporting layer enables the production of an organic EL device which can be driven at an extremely low voltage and shows high current efficiency.

### INDUSTRIAL APPLICABILITY

As described above in detail, the organic EL device using the nitrogen-containing heterocyclic derivative of the present invention shows high luminous brightness and high luminous efficiency even at a low voltage as compared with a conventional device.

Accordingly, the organic EL device using the nitrogen-containing heterocyclic derivative of the present invention is extremely useful as an organic EL device having high practicality.

1. A nitrogen-containing heterocyclic derivative, which is represented by the following formula (1): 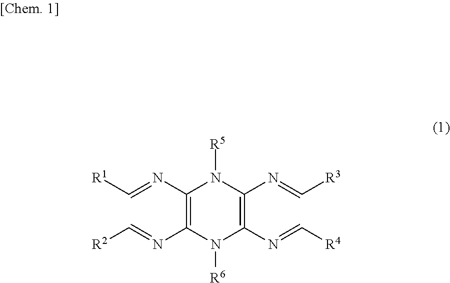
where:
R¹ to R⁴ each independently represent a hydrogen atom, a substituted or unsubstituted aryl group having 6 to 60 ring atoms, a substituted or unsubstituted heteroaryl group having 5 to 60 ring atoms, a substituted or unsubstituted alkyl group having 1 to 50 carbon atoms, a substituted or unsubstituted cycloalkyl group having 3 to 50 carbon atoms, a substituted or unsubstituted haloalkyl group having 1 to 50 carbon atoms, a substituted or unsubstituted aryloxycarbonyl group having 6 to 60 ring atoms, a substituted or unsubstituted alkoxycarbonyl group having 1 to 50 carbon atoms, a substituted or unsubstituted carbamoyl group, a substituted or unsubstituted arylcarbonyl group having 6 to 60 ring atoms, a substituted or unsubstituted alkylcarbonyl group having 1 to 50 carbon atoms, a substituted or unsubstituted arylsulfonyl group having 6 to 60 ring atoms, a substituted or unsubstituted alkylsulfonyl group having 1 to 50 carbon atoms, a substituted or unsubstituted arylsulfinyl group having 6 to 60 ring atoms, a substituted or unsubstituted alkylsulfinyl group having 1 to 50 carbon atoms, a halogen atom, a cyano group, or a nitro group, and adjacent groups of R¹ to R⁴ may be bonded to each other to form a ring structure; and
R⁵ and R⁶ each independently represent a substituted or unsubstituted aryl group having 6 to 60 ring atoms, a substituted or unsubstituted heteroaryl group having 5 to 60 ring atoms, a substituted or unsubstituted alkyl group having 1 to 50 carbon atoms, a substituted or unsubstituted cycloalkyl group having 3 to 50 carbon atoms, or a substituted or unsubstituted haloalkyl group having 1 to 50 carbon atoms.

2. The nitrogen-containing heterocyclic derivative according to claim 1, wherein at least one of R¹ to R⁴ in the formula (1) represents an electron-withdrawing substituent selected from a substituted or unsubstituted haloalkyl group having 1 to 50 carbon atoms, a substituted or unsubstituted aryloxycarbonyl group having 6 to 60 ring atoms, a substituted or unsubstituted alkoxycarbonyl group having 1 to 50 carbon atoms, a substituted or unsubstituted carbamoyl group, a substituted or unsubstituted arylcarbonyl group having 6 to 60 ring atoms, a substituted or unsubstituted alkylcarbonyl group having 1 to 50 carbon atoms, a substituted or unsubstituted arylsulfonyl group having 6 to 60 ring atoms, a substituted or unsubstituted alkylsulfonyl group having 1 to 50 carbon atoms, a substituted or unsubstituted arylsulfinyl group having 6 to 60 ring atoms, a substituted or unsubstituted alkylsulfinyl group having 1 to 50 carbon atoms, a cyano group, and a nitro group.

3. The nitrogen-containing heterocyclic derivative according to claim 1, wherein the nitrogen-containing heterocyclic derivative represented by the formula (1) is represented by the following formula (2): 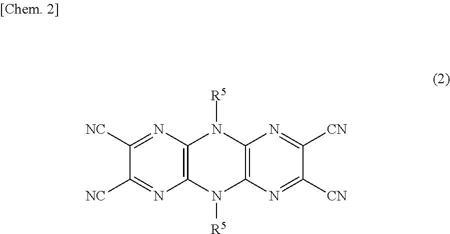
where R⁵ and R⁶ each have the same meaning as that described above.

4. The nitrogen-containing heterocyclic derivative according to claim 1, wherein the nitrogen-containing heterocyclic derivative represented by the formula (1) is represented by the following formula (3-a), (3-b), (3-c), (3-d), (3-e), (3-f), (3-g), or (3-h): 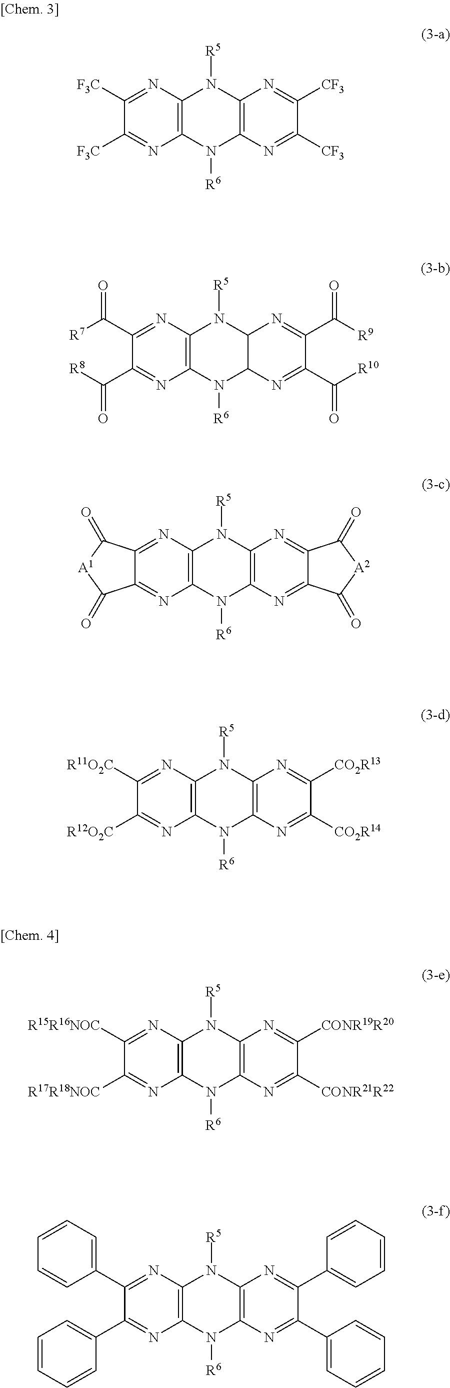 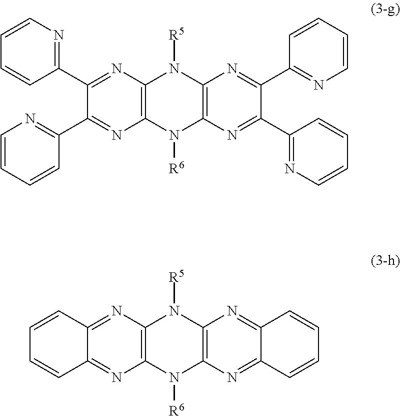
where:
R⁷ to R¹⁰ each independently represent a substituted or unsubstituted aryl group having 6 to 60 ring atoms, a substituted or unsubstituted heteroaryl group having 5 to 60 ring atoms, a substituted or unsubstituted alkyl group having 1 to 50 carbon atoms, a substituted or unsubstituted cycloalkyl group having 3 to 50 carbon atoms, or a substituted or unsubstituted haloalkyl group having 1 to 50 carbon atoms, and adjacent substituents may be bonded to each other to form a ring structure;
A¹ and A² each independently represent an oxygen atom or —NR′—, and R′ represents a substituted or unsubstituted arylene group having 6 to 60 ring atoms, a substituted or unsubstituted heteroarylene group having 5 to 60 ring atoms, a substituted or unsubstituted alkylene group having 1 to 50 carbon atoms, a substituted or unsubstituted cycloalkylene group having 3 to 50 carbon atoms, or a substituted or unsubstituted haloalkylene group having 1 to 50 carbon atoms;
R¹¹ to R¹⁴ each independently represent a substituted or unsubstituted aryl group having 6 to 60 ring atoms, a substituted or unsubstituted heteroaryl group having 5 to 60 ring atoms, a substituted or unsubstituted alkyl group having 1 to 50 carbon atoms, a substituted or unsubstituted cycloalkyl group having 3 to 50 carbon atoms, or a substituted or unsubstituted haloalkyl group having 1 to 50 carbon atoms; and
R¹⁵ to R²² each independently represent a hydrogen atom, a substituted or unsubstituted aryl group having 6 to 60 ring atoms, a substituted or unsubstituted heteroaryl group having 5 to 60 ring atoms, a substituted or unsubstituted alkyl group having 1 to 50 carbon atoms, a substituted or unsubstituted cycloalkyl group having 3 to 50 carbon atoms, or a substituted or unsubstituted haloalkyl group having 1 to 50 carbon atoms.

5. The nitrogen-containing heterocyclic derivative according to claim 1, wherein the nitrogen-containing heterocyclic derivative represented by the formula (1) is represented by the following formula (4-a) or (4-b): 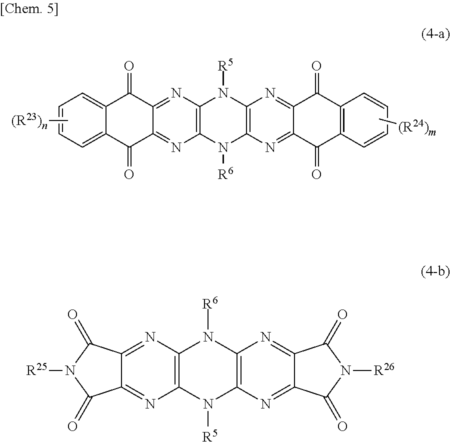
where:
R²³ to R²⁶ each independently represent a substituted or unsubstituted aryl group having 6 to 60 ring atoms, a substituted or unsubstituted heteroaryl group having 5 to 60 ring atoms, a substituted or unsubstituted alkyl group having 1 to 50 carbon atoms, a substituted or unsubstituted cycloalkyl group having 3 to 50 carbon atoms, or a substituted or unsubstituted haloalkyl group having 1 to 50 carbon atoms; and
n and m each represent an integer of 1 to 4.

6. A hole injecting material or hole transporting material for an organic electroluminescence device, comprising the nitrogen-containing heterocyclic derivative according to claim 1.

7. A light emitting material for an organic electroluminescence device, comprising the nitrogen-containing heterocyclic derivative according to claim 1.

8. An electron injecting material or electron transporting material for an organic electroluminescence device, comprising the nitrogen-containing heterocyclic derivative according to claim 1.

9. An organic electroluminescence device, comprising one or a plurality of organic layers interposed between a cathode and an anode, wherein at least one layer of the organic layers contains the nitrogen-containing heterocyclic derivative according to claim 1.

10. The organic electroluminescence device according to claim 9, wherein the organic layer containing the nitrogen-containing heterocyclic derivative comprises a hole injecting layer or a hole transporting layer.

11. The organic electroluminescence device according to claim 10, wherein the hole injecting layer or the hole transporting layer further contains a hole injecting substance selected from the group consisting of a phthalocyanine copper complex compound, an oligothiophene, an arylamine-based compound, and a polycyclic aromatic compound.

12. The organic electroluminescence device according to claim 9, wherein the organic layer containing the nitrogen-containing heterocyclic derivative comprises a light emitting layer.

13. The organic electroluminescence device according to claim 9, wherein the organic layer containing the nitrogen-containing heterocyclic derivative comprises an electron injecting layer or an electron transporting layer.

14. The organic electroluminescence device according to claim 13, wherein the electron injecting layer or the electron transporting layer further contains a reduction-causing dopant.

15. The organic electroluminescence device according to claim 14, wherein the reduction-causing dopant comprises one kind or two or more kinds of substances selected from the group consisting of alkali metals, alkaline earth metals, rare earth metals, oxides of alkali metals, halides of alkali metals, oxides of alkaline earth metals, halides of alkaline earth metals, oxides of rare earth metals, halides of rare earth metals, organic complexes of alkali metals, organic complexes of alkaline earth metals, and organic complexes of rare earth metals.

16. An apparatus, comprising the organic electroluminescence device according to claim 9.
